SEQUENCE LISTING (13 sequences)

SEQ ID NO: 1 - protein, 11 aa, Homo sapiens
RASQSVSSNLA

SEQ ID NO: 2 - protein, 7 aa, Homo sapiens
DASTRAT

SEQ ID NO: 3 - protein, 9 aa, Homo sapiens
QQYDNWPLT

SEQ ID NO: 4 - protein, 5 aa, Homo sapiens
RYGIS

SEQ ID NO: 5 - protein, 17 aa, Homo sapiens
WISTYSGNTNYAQKLQG

SEQ ID NO: 6 - protein, 7 aa, Homo sapiens
RQLYFDY

SEQ ID NO: 7 - protein, 107 aa, Homo sapiens
EIVMTQSPATLSVSPGERATLSCRASQSVSSNLAWFQQKPGQAPRPLIYDASTRATGVPARFSGSGSGTDFTLTISSLQSEDFAVYYCQQYDNWPLTFGGGTKVEIK

SEQ ID NO: 8 - protein, 116 aa, Homo sapiens
QVQLVQSGAEVKKPGASVKVSCKASGYTFTRYGISWVRQAPGQGLEWMGWISTYSGNTNYAQKLQGRVTMTTDTSTSTAYMELRSLRSDDTAVYYCARRQLYFDYWGQGTLVTVSS

SEQ ID NO: 9 - protein, 214 aa, Homo sapiens
EIVMTQSPATLSVSPGERATLSCRASQSVSSNLAWFQQKPGQAPRPLIYDASTRATGVPARFSGSGSGTDFTLTISSLQSEDFAVYYCQQYDNWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 461 aa, Homo sapiens
MEWTWRVLFLVAAATGAHSQVQLVQSGAEVKKPGASVKVSCKASGYTFTRYGISWVRQAPGQGLEWMGWISTYSGNTNYAQKLQGRVTMTTDTSTSTAYMELRSLRSDDTAVYYCARRQLYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 11 - DNA, 741 bp, Homo sapiens
gtcgacgtttaaacgccgccaccatggaagcgccggcgcagcttctcttcctcctgctactctggctcccagataccactggagaaatagtgatgacgcagtctccagccaccctgtctgtgtctcctggggaaagagccaccctctcctgcagggccagtcagagtgttagcagcaacttagcctggttccagcagaaacctggccaggctcccaggcccctcatctatgatgcatccaccagggccactggtgtcccagccaggttcagtggcagtgggtctgggacagacttcactctcaccatcagcagcctgcagtctgaagattttgcagtttattactgtcagcagtatgataactggccgctcactttcggcggagggaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaggatccgcggccgc

SEQ ID NO: 12 - DNA, 1409 bp, Homo sapiens
gtcgacgccgccaccatggagtggacctggagggtccttttcttggtggcagcagcaacaggtgcccactcccaggttcagctggtgcagtctggagctgaggtgaagaagcctggggcctcagtgaaggtctcctgcaaggcttctggttacacctttaccagatatggtatcagctgggtgcgacaggcccctggacaagggcttgagtggatgggatggatcagcacttacagtggtaacacaaactatgcacagaagctccagggcagagtcaccatgaccacagacacatccacgagcacagcctacatggagctgaggagcctgagatctgacgacacggccgtgtattactgtgcgagacggcagctttactttgactactggggccagggaaccctggtcaccgtctcctcagctagcaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgagcggccgc

SEQ ID NO: 13 - protein, 866 aa, Homo sapiens
MGAARSPPSAVPGPLLGLLLLLLGVLAPGGASLRLLDHRALVCSQPGLNCTVKNSTCLDDSWIHPRNLTPSSPKDLQIQLHFAHTQQGDLFPVAHIEWTLQTDASILYLEGAELSVLQLNTNERLCVRFEFLSKLRHHHRRWRFTFSHFVVDPDQEYEVTVHHLPKPIPDGDPNHQSKNFLVPDCEHARMKVTTPCMSSGSLWDPNITVETLEAHQLRVSFTLWNESTHYQILLTSFPHMENHSCFEHMHHIPAPRPEEFHQRSNVTLTLRNLKGCCRHQVQIQPFFSSCLNDCLRHSATVSCPEMPDTPEPIPDYMPLWVYWFITGISILLVGSVILLIVCMTWRLAGPGSEKYSDDTKYTDGLPAADLIPPPLKPRKVWIIYSADHPLYVDVVLKFAQFLLTACGTEVALDLLEEQAISEAGVMTWVGRQKQEMVESNSKIIVLCSRGTRAKWQALLGRGAPVRLRCDHGKPVGDLFTAAMNMILPDFKRPACFGTYVVCYFSEVSCDGDVPDLFGAAPRYPLMDRFEEVYFRIQDLEMFQPGRMHRVGELSGDNYLRSPGGRQLRAALDRFRDWQVRCPDWFECENLYSADDQDAPSLDEEVFEEPLLPPGTGIVKRAPLVREPGSQACLAIDPLVGEEGGAAVAKLEPHLQPRGQPAPQPLHTLVLAAEEGALVAAVEPGPLADGAAVRLALAGEGEACPLLGSPGAGRNSVLFLPVDPEDSPLGSSTPMASPDLLPEDVREHLEGLMLSLFEQSLSCQAQGGCSRPAMVLTDPHTPYEEEQRQSVQSDQGYISRSSPQPPEGLTEMEEEEEEEQDPGKPALPLSPEDLESLRSLQRQLLFRQLQKNSGWDTMGSESEGPSA

(19) United States
(12) Patent Application Publication
(10) Pub. No.: US 2017/0174772 A1
(43) Pub. Date: Jun. 22, 2017

(54) METHODS OF TREATING NAIL AND SCALP PSORIASIS

(71) Applicant: KIRIN-AMGEN, INC., THOUSAND OAKS, CA (US)

(72) Inventors: Ajay Nirula, Westlake Village, CA (US); Hidemi Nakagawa, Tokyo (JP); Kenji Ohtaki, Tokyo (JP); Hiroki Matsudo, Tokyo (JP); Paul Klekotka, Newbury Park, CA (US); Gregory Kricorian, Thousand Oaks, CA (US)

(73) Assignee: Kirin-Amgen, Inc., Thosand Oaks, CA (US)

(21) Appl. No.: 15/129,811

(22) PCT Filed: Mar. 19, 2015
(86) PCT No.: PCT/US2015/021613

§ 371 Date: Sep. 27, 2016

Related U.S. Application Data
(60) Provisional application No. 62/041,879, filed on Aug. 26, 2014.
(60) Provisional application No. 62/031,850, filed on Jul. 31, 2014.
(60) Provisional application No. 61/972,638, filed on Mar. 31, 2014.

(51) Int. Cl. C07K 16/28

(52) U.S. Cl. CPC C07K 16/2866; C07K 2317/76; C07K 2317/565; C07K 2317/21; A61K 2039/505

(57) ABSTRACT

The present invention relates to a therapeutic agent for nail and scalp psoriasis comprising an IL-17 Receptor A (IL-17RA or IL-17R) antigen binding proteins, such as monoclonal antibodies that bind IL-17RA, and method of using the same.

The present application claims priority benefit to U.S. Provisional Application No. 61/972,638, filed Mar. 31, 2014, U.S. Provisional Patent Application No. 62/031,850 filed Jul. 31, 2014, and U.S. Provisional Patent Application No. 62/041,879 filed Aug. 26, 2014, all of which are incorporated by reference herein in their entirety.

### FIELD OF THE INVENTION

The present invention relates to a therapeutic agent for nail psoriasis and scalp psoriasis comprising an IL-17 receptor A (IL 17RA or IL-17R) antigen binding proteins such as a monoclonal antibody or an antibody fragment thereof as an active ingredient. The present invention relates to methods of treating nail and scalp psoriasis with IL-17RA antigen binding proteins, such as a monoclonal antibodies or antibody fragment thererof.

### BACKGROUND

Psoriasis is a chronic and debilitating immune-related inflammatory disease that can involve the scalp and nail beds. This disease causes significant social stigma for patients and also is a major economic burden. In some cases, nail or scalp psoriasis may be the only affected area and these patients are unlikely to meet the requirements for systemic or biologic therapies.

Psoriasis is thought to affect the scalp in two ways: psoriatic alopecia, without any scaliness, and regular scalp psoriasis, with typical epidermal involvement (Hermanns-Le et al., J. Biomed. Biotech. 2012: 1-6, 2012). Scalp psoriasis is very difficult to treat, and many times it is disabling for the patient as it causes many quality of life issues. Many topical therapies for scalp psoriasis are difficult and unpleasant to apply and as a result patient compliance and adherence to treatment regimens is often reduced. In addition, as the scalp qualifies as a small percentage of body surface area, many patients suffering from scalp psoriasis do not qualify for treatment with biological agents. The typical treatment scheme is topical, including tar and salicylic acid, followed by photoptherapy, and then systemic therapies, such as methrotrexate and acitretin. The systemic therapies may cause further hair loss, and thereby exacerbate the hair loss associated with scalp psoriasis itself. (Kircik and Kumar, J. Drugs Dermolog. 9: s101-s105, 2010).

Nail psoriasis is often overlooked as the nails are largely asymptomatic in the early stages of disease. The current treatments for nail psoriasis are often poorly efficacious and associated with undesirable systemic effects. In addition, many of the treatments are time consuming and impractical for the patient. Due to the anatomical structure of the nail unit, it is generally difficult to achieve sufficient concentrations of adsorptive treatment agents in the involved nail, nail bed or matrix by topical administration. (Wozel, Clin. Derm. 26:448-459, 2008), while the small amount of surface area affected may not qualify a patient for systemic or biologic treatment.

IL-17A is an inflammatory cytokine initially identified as a transcript selectively expressed by activated T cells. IL-17RA is a ubiquitously expressed and shown to bind IL-17A with an affinity of approximately 0.5 nM (Yao et al., 1995, Immunity 3:811-821). Five additional IL-17-like ligands (IL-17B-IL-17F) and four additional IL-17RA-like receptors (IL-17RB-IL-17RE) have been identified (Kolls and Linden, 2004, Immunity 21:467-476).

IL-17A and IL-17F bind and activate IL-17RA. IL-17RA has been shown to be important in regulating immune responses. Activation of the IL-17RA leads to production of cytokines, chemokines, growth factors, and other proteins that contribute to the symptoms and/or pathology of numerous diseases. IL-17A is an inflammatory cytokine that induces the production of cytokines and other mediators leading to diseases and physiological effects such as inflammation, cartilage degradation, and bone resorption. IL-17A also plays a role in a number of inflammatory conditions including arthritis (rheumatoid arthritis), psoriasis, inflammatory bowel disease, multiple sclerosis, and asthma. (Li et al., 2004, Huazhong Univ. Sci. Technolog. Med. Sci. 24:294-296; Fujino et al., 2003, Gut. 52:65-70; Kauffman et al., 2004, J. Invest. Dermatol. 123:1037-1044; Mannon et al., 2004, N. Engl. J Med. 351:2069-2079; Matusevicius et al., 1999, Mult Scler 5, 101-104; Linden et al., Eur Respir J. 2000 May;15(5):973-7; Molet et al., 2001, J. Allergy Clin. Immunol. 108:430-438). Recent studies have suggested that IL-17F plays a role in the induction of inflammatory responses (Oda et al., 2006, American J. Resp. Crit. Care Medicine, Jan. 15, 2006; Numasaki et al., 2004, Immunol Lett. 95:97-104).

There is currently a long-standing unmet need for safe and effective treatments for scalp and nail psoriasis. The invention contemplates treatments of scalp and nail psoriasis using IL-17 Receptor A (IL-17RA or IL-17R) antigen binding proteins, such as the monoclonal antibody brodalumab, as well as other agents that inhibit the IL-17 signaling axis.

### SUMMARY OF INVENTION

The invention provides methods of treating nail or scalp psoriasis or therapeutic agents for nail or scalp psoriasis comprising administering an IL-17RA antigen binding protein, such as a monoclonal antibody or fragment thereof that specifically binds to the IL-17RA. The invention provides methods of treating nail or scalp psoriasis or therapeutic agents for nail or scalp psoriasis comprising administering monoclonal antibodies or fragment thereof that specifically bind to the IL-17RA and have antagonistic activities. Such antagonistic activities include inhibiting binding of IL-17A to the IL-17RA. An exemplary monoclonal antibody, known as AM-14 or brodalumab, comprises a light chain variable domain comprising the amino acid sequence of SEQ ID NO:7 and a heavy chain variable domain comprising the amino acid sequence of SEQ ID NO:8. The full length light chain of brodalumab is set out as the amino acid sequence of SEQ ID NO: 9 and the full length heavy chain of brodalumab is set out as the amino acid sequence of SEQ ID NO: 10. The study described herein demonstrates that brodalumab is more efficacious than a placebo in treating scalp or nail psoriasis after 12 weeks of treatment.

The invention also provides for use of an antibody or fragment thereof for the preparation of a medicament for the treatment of nail or scalp psoriasis, wherein the antibody or fragment thereof the specifically binds to IL-17RA and has an antagonistic activity. The invention further provides for a composition for use in the treatment of nail or scalp psoriasis, wherein the composition comprises an antibody or fragment thereof that specifically binds to IL-17RA and has an antagonistic activity. Such antagonistic activity includes inhibiting binding of IL-17A to IL-17RA.

An aspect of the invention is methods of treating nail or scalp psoriasis, or therapeutic agents for nail or scalp psoriasis, comprising administering to a patient having nail or scalp psoriasis a composition comprising an antibody or fragment thereof selected from: an antibody, comprising a light chain variable domain comprising the amino acid sequence of SEQ ID NO:7 and a heavy chain variable domain comprising the amino acid sequence of SEQ ID NO: 8; an antibody comprising the full length light chain of SEQ ID NO: 9 and a full length heavy chain of SEQ ID NO: 10 and an antibody, comprising a light chain CDR1 comprising the amino acid sequence of SEQ ID NO:1, a light chain CDR2 comprising the amino acid sequence of SEQ ID NO:2, a light chain CDR3 comprising the amino acid sequence of SEQ ID NO:3, a heavy chain CDR1 comprising the amino acid sequence of SEQ ID NO:4, a heavy chain CDR2 comprising the amino acid sequence of SEQ ID NO:5, and a heavy chain CDR3 comprising the amino acid sequence of SEQ ID NO:6, wherein said antibody specifically binds to human IL-17RA. For example in the methods of treating nail or scalp psoriasis or therapeutic agents for nail and scalp psoriasis, the monoclonal antibody is a human monoclonal antibody that specifically binds to human IL-17RA and inhibits the binding of IL-17A to said IL-17RA.

Another aspect of the invention is use of an antibody or fragment thereof for the preparation of a medicament for the treatment of nail or scalp psoriasis, wherein the composition comprises an antibody or fragment thereof selected from: an antibody, comprising a light chain variable domain comprising the amino acid sequence of SEQ ID NO:7 and a heavy chain variable domain comprising the amino acid sequence of SEQ ID NO: 8; an antibody comprising the full length light chain of SEQ ID NO: 9 and a full length heavy chain of SEQ ID NO: 10 and an antibody, comprising a light chain CDR1 comprising the amino acid sequence of SEQ ID NO:1, a light chain CDR2 comprising the amino acid sequence of SEQ ID NO:2, a light chain CDR3 comprising the amino acid sequence of SEQ ID NO:3, a heavy chain CDR1 comprising the amino acid sequence of SEQ ID NO:4, a heavy chain CDR2 comprising the amino acid sequence of SEQ ID NO:5, and a heavy chain CDR3 comprising the amino acid sequence of SEQ ID NO:6, wherein said antibody specifically binds to human IL-17RA. For example in the uses of treating nail or scalp psoriasis, the antibody is a human monoclonal antibody that specifically binds to human IL-17RA and inhibits the binding of IL-17A to said IL-17RA.

In another aspect of the invention, the invention provide for compositions for use in the treatment or nail or scalp psoriasis comprising an antibody or fragment thereof, wherein the composition comprises an antibody or fragment thereof selected from: an antibody, comprising a light chain variable domain comprising the amino acid sequence of SEQ ID NO:7 and a heavy chain variable domain comprising the amino acid sequence of SEQ ID NO: 8; an antibody comprising the full length light chain of SEQ ID NO: 9 and a full length heavy chain of SEQ ID NO: 10 and an antibody, comprising a light chain CDR1 comprising the amino acid sequence of SEQ ID NO:1, a light chain CDR2 comprising the amino acid sequence of SEQ ID NO:2, a light chain CDR3 comprising the amino acid sequence of SEQ ID NO:3, a heavy chain CDR1 comprising the amino acid sequence of SEQ ID NO:4, a heavy chain CDR2 comprising the amino acid sequence of SEQ ID NO:5, and a heavy chain CDR3 comprising the amino acid sequence of SEQ ID NO:6, wherein said antibody specifically binds to human IL-17RA. For example, in the composition for treating nail or scalp psoriasis, the antibody is a human monoclonal antibody that specifically binds to human IL-17RA and inhibits the binding of IL-17A to said IL-17RA.

Nail psoriasis in the present invention means psoriasis developing on the nail in psoriasis patients and includes one or more occurrence of pitting, leukonychia, red spots in lunula, nail plate crumbling, oil drop (salmon patch) discoloration, onycholysis, opacity, thickening, nail bed hyperkeratosis, and splinter hemorrhages. In addition, scalp psoriasis means psoriasis developing on the scalp in psoriasis patients. The kinds of psoriasis of the nail and scalp include psoriasis vulgaris, psoriasis arthropica, pustular psoriasis, psoriatic erythroderma, guttate psoriasis, palmoplantar psoriasis, and plaque psoriasis and the like. A patient having nail psoriasis or scalp psoriasis in the present invention may also have developed psoriasis on other areas of their body; for example, the patient may have psoriasis vulgaris, psoriasis arthropica, pustular psoriasis, psoriatic erythroderma, guttate psoriasis and plaque psoriasis and the like on an area of the body in addition to having one or more of these types of psoriasis on the nails or scalp. Alternatively, the patient may have psoriasis primarily or only on the nail or scalp.

In the methods, uses, compositions or therapeutic agents of the invention, the severity of nail psoriasis is assessed using the Nail Psoriasis Severity Index (NAPSI) score or the modified NAPSI (mNAPSI). The NAPSI scale, first proposed by Rich and Scher (J. AM. Acad. Dermalol. 49(2): 206-12, 2003), is determined by first dividing the nail with imaginary horizontal and vertical lines into 4 quarters. The following eight clinical features of nail psoriasis are then scored based on the number of quarters in which the feature is present (0 to 4) to arrive at a NAPSI score of 0 to 32 for each nail: pitting, leukonychia, red spots in lunula, nail plate crumbling, oil drop (salmon patch) discoloration, onycholysis, nail bed hyperkeratosis, and splinter hemorrhages. Onycholysis with a red border is more specific for nail psoriasis than onycholysos without erythema. The severity of nail psoriasis is assessed using the modified NAPSI (mNAPSI) scale, which takes into account the degree of gradation of each parameter from 0 to 3 (0=none, 1=mild, 2=moderate and 3=severe). For example, patients with a NAPSI score of at least 6 or mNAPSI score of at least 2 or 3 for at least one affected nail were considered to have moderate to severe nail psoriasis.

In the methods, uses, compositions or therapeutic agents of the invention, the severity of scalp psoriasis is assessed using the Psoriasis Scalp Severity Index (PSSI) score and/or the affected Scalp Surface Area (SSA) score. The PSSI is a scalp-specific modification of the Psoriasis Area Severity Index (PASI), based on the extent of involvement and the severity of erthyema, induration and desquamation. The SSA numerical score (0% to 100%) measures the assessment of the proportion of the subject's total SSA involved with psoriasis. Patients with a PSSI≧15 and an SSA≧30% were considered to have moderate to severe scalp psoriasis.

The methods for treating nail or scalp psoriasis in the present invention are treatment methods capable of reducing the degree of severity of psoriasis, which is developed on the nail or the scalp. In addition, the therapeutic agents for nail or scalp psoriasis in the present invention are therapeutic agents capable of reducing the degree of severity of psoriasis which developed on the nail or the scalp. In addition, the treatment methods of the present invention are also treatment methods capable of improving symptoms of psoriasis developed on the nail or scalp. In addition, the therapeutic agents of the present invention are therapeutic agents capable of improving symptoms of psoriasis developed on the nail or scalp.

An improvement in symptoms of psoriasis developed on the nail (nail psoriasis) refers to a decrease in numerical value of the NAPSI score or mNAPSI score after the administration of an IL-17RA antigen binding protein of the invention relative to before the administration, and preferably the numerical value of the score is decreased in a group to which the IL-17RA antigen binding protein in the invention is administered in comparison to a placebo administered group. In addition, the improvement refers to a decrease in the mean percent change in the NAPSI score or mNAPSI score from baseline, and preferably the mean percent change in the NAPSI score or mNAPSI score from baseline is decreased in the group to which the IL-17RA antigen binding protein is administered in comparison to the placebo administered group.

In addition, the improvement refers to an increase in the NAPSI or mNAPSI score mean percent improvement from the initial administration (baseline), and preferably the NAPSI or mNAPSI mean percent improvement from baseline is increased in the group to which the IL-17RA antigen binding protein is administered in comparison to the placebo administered group.

An improvement in symptoms of psoriasis developed on the scalp (scalp psoriasis) refers to a decrease in numerical value of the PSSI score after the administration of the IL-17RA antigen binding protein relative to before the administration, and preferably the numerical value of the score is decreased in a group to which the IL-17RA antigen binding protein is administered in comparison to a placebo administered group (baseline). In addition, the improvement refers to a decrease in the mean percent change in the PSSI score from baseline, and preferably the mean percent change in the PSSI score from baseline is decreased in the group to which the IL-17RA antigen binding protein is administered in comparison to the placebo administered group.

In addition, the improvement refers to an increase in the PSSI mean percent improvement from the initial administration (baseline), and preferably the PSSI mean percent improvement from baseline is increased in the group to which the IL-17RA antigen binding protein is administered in comparison to the placebo administered group.

The mean percent change in the NAPSI or PSSI score from baseline is calculated as follows:

The mean percent change in the NAPSI, mNAPSI or PSSI score from baseline (%)=(the NAPSI, mNAPSI or PSSI score after administration of the IL-17RA antigen binding protein/the NAPSI, mNAPSI or PSSI score on baseline−1)×100. Formula 1

The NAPSI, mNAPSI or PSSI percent improvement from baseline is calculated as follows:

The NAPSI, mNAPSI or PSSI percent improvement from baseline (%)=(1−the NAPSI, mNAPSI or PSSI score after administration of the IL-17RA antigen binding protein/the NAPSI, mNAPSI or PSSI score on baseline)×100.

For example, the baseline is the initial administration day.

In addition, the improvement refers to an increase in PSSI-75 or PSSI-100, and preferably the PSSI-75 or PSSI-100 is increased in the group to which the IL-17RA antigen binding protein is administered in comparison to the placebo administered group. PSSI-75 or PSSI-100 means percent of patients who achieve 75% or 100% of the PSSI mean percent improvement, respectively.

The placebo administered group may be any drug as long as a drug does not contain an active ingredient; however, specific examples include a solvent of an antibody formulation and the like. The administration may be single administration or multiple administrations (hereinafter, described as “continuous administration”).

The therapeutic agent of the present invention includes a therapeutic agent in which the mean percent change in NAPSI or mNAPSI from baseline is reduced by 20% or more, 30% or more, 40% or more, 50% or more, 60% or more, 70% or more, 80% or more, 90% or more or 100%. Further, the therapeutic agent may include a therapeutic agent in which the mean percent change in the NAPSI or mNAPSI from baseline is reduced by 30% or more, 40% or more, 50% or more, 60% or more, 70% or more, 80% or more, 90% or more or 100% in the group to which the therapeutic agent is administered, with respect to the placebo administration group.

The therapeutic agent of the present invention includes a therapeutic agent in which the mean percent change in PSSI from baseline is reduced by 30% or more, 40% or more, 50% or more, 60% or more, 70% or more, 80% or more, 90% or more or 100%. In addition, the therapeutic agent may include a therapeutic agent in which the mean percent change in the PSSI from baseline is reduced by 20% or more, 30% or more, 40% or more, 50% or more, 60% or more, 70% or more, 80% or more, 90% or more or 100% in the group to which the therapeutic agent is administered, with respect to the placebo administered group.

The treatment method of the present invention includes a treatment method in which the mean percent change in NAPSI or mNAPSI from baseline is reduced by 20% or more, 30% or more, 40% or more, 50% or more, 60% or more, 70% or more, 80% or more, 90% or more or 100% in the group to which the antibody or the antibody fragment thereof is administered. In addition, the treatment method may include a treatment method in which the mean percent change in the NAPSI or mNAPSI is from baseline reduced by 30% or more, 40% or more, 50% or more, 60% or more, 70% or more, 80% or more, 90% or more or 100% in the group to which the antibody or the antibody fragment thereof is administered, with respect to the placebo administered group.

The treatment method of the present invention includes a treatment method in which the mean percent change in PSSI from baseline after the administration is reduced by 30% or more, 40% or more, 50% or more, 60% or more, 70% or more, 80% or more, 90% or more or 100% in the group to which the antibody or the antibody fragment thereof is administered. In addition, the treatment method may include a treatment method in which the mean percent change in the PSSI from baseline is reduced by 20% or more, 30% or more, 40% or more, 50% or more, 60% or more, 70% or more, 80% or more, 90% or more or 100% in the group to which the antibody or the antibody fragment thereof is administered, with respect to the placebo administered group.

Any of the therapeutic agents, medicaments or compositions of the invention may be administered to induce a mean percent change in NAPSI or mNAPSI from baseline is reduced by 20% or more, 30% or more, 40% or more, 50% or more, 60% or more, 70% or more, 80% or more, 90% or more or 100% in the group to which the antibody or the antibody fragment thereof is administered. In addition, any of the therapeutic agents, medicaments or compositions of the invention may be administered to induce a mean percent change in the NAPSI or mNAPSI is from baseline reduced by 30% or more, 40% or more, 50% or more, 60% or more, 70% or more, 80% or more, 90% or more or 100% in the group to which the antibody or the antibody fragment thereof is administered, with respect to the placebo administered group.

Any of the therapeutic agents, medicaments or compositions of the invention may be administered to induce a mean percent change in PSSI from baseline after the administration is reduced by 30% or more, 40% or more, 50% or more, 60% or more, 70% or more, 80% or more, 90% or more or 100% in the group to which the antibody or the antibody fragment thereof is administered. In addition, any of the therapeutic agents, medicaments or compositions of the invention may be administered to induce a mean percent change in the PSSI from baseline is reduced by 20% or more, 30% or more, 40% or more, 50% or more, 60% or more, 70% or more, 80% or more, 90% or more or 100% in the group to which the antibody or the antibody fragment thereof is administered, with respect to the placebo administered group.

In one embodiment, the patient treated with the preceding method or the patient administered a therapeutic agent, medicament or composition of the invention has 10% or greater body surface area affected by moderate to severe plaque psoriasis prior to treatment. Moderate to severe nail psoriasis includes patients with a NAPSI score of at least 6 or mNAPSI score of at least 2 or 3 on one or more nails. For example, the patient with moderate to severe psoriasis has the presence of at least two of the following clinical features in at least one quarter of a nail: pitting, leukonychia, red spots in lunula, nail plate crumbling, oil drop (salmon patch) discoloration, onycholysis, nail bed hyperkeratosis, splinter hemorrhages or has at least one of these clinical features in more than one quarter of the one or more nails. Moderate to severe scalp psoriasis include patients having a PSSI score of at least 15 and/or a SSA score of at least 30%.

Another aspect of the invention provides for methods of treating nail psoriasis, comprising administering to a patient having a pretreatment NAPSI score of at least 6 or pretreatment mNAPSI score of at least 2 or 3 for at least one affected nail, a composition comprising an human antibody or fragment thereof selected from: an antibody, comprising a light chain variable domain comprising the amino acid sequence of SEQ ID NO:7 and a heavy chain variable domain comprising the amino acid sequence of SEQ ID NO:8; an antibody comprising the full length light chain of SEQ ID NO: 9 and a full length heavy chain of SEQ ID NO: 10 and an antibody, comprising a light chain CDR1 comprising the amino acid sequence of SEQ ID NO:1, a light chain CDR2 comprising the amino acid sequence of SEQ ID NO:2, a light chain CDR3 comprising the amino acid sequence of SEQ ID NO:3, a heavy chain CDR1 comprising the amino acid sequence of SEQ ID NO:4, a heavy chain CDR2 comprising the amino acid sequence of SEQ ID NO:5, and a heavy chain CDR3 comprising the amino acid sequence of SEQ ID NO:6, wherein said human monoclonal antibody specifically binds to human IL-17 receptor A and inhibits the binding of IL-17A to said IL-17 receptor A, wherein the compositions is administered at a dose effective to reduce and maintain a NAPSI score 6 or less or mNAPSI of 3 or less on an affected nail.

In any of the preceding methods, the patient has about 20% or greater body surface area affected by psoriasis, such as plaque psoriasis, or the patient has about 30% or greater body surface area affected by psoriasis or the patient has about 40% or greater body surface area affected by psoriasis, or the patient has about 50% or greater body surface area affected by psoriasis, or the patient has about 60% or greater body surface area affected by psoriasis, or the patient has about 70% or less body surface area affected by psoriasis or the patient has about 80% or greater body surface area affected by psoriasis, or the patient has about 90% or greater body surface area affected by psoriasis.

In another aspect, the invention provides for a therapeutic agent, medicament and composition for treating nail psoriasis in a patient having a pretreatment NAPSI score of at least 6 or pretreatment mNAPSI scores of at least 2 or 3 for at least one affected nail, a composition comprising an antibody or fragment thereof selected from: an antibody, comprising a light chain variable domain comprising the amino acid sequence of SEQ ID NO:7 and a heavy chain variable domain comprising the amino acid sequence of SEQ ID NO:8; an antibody comprising the full length light chain of SEQ ID NO: 9 and a full length heavy chain of SEQ ID NO: 10 and an antibody, comprising a light chain CDR1 comprising the amino acid sequence of SEQ ID NO:1, a light chain CDR2 comprising the amino acid sequence of SEQ ID NO:2, a light chain CDR3 comprising the amino acid sequence of SEQ ID NO:3, a heavy chain CDR1 comprising the amino acid sequence of SEQ ID NO:4, a heavy chain CDR2 comprising the amino acid sequence of SEQ ID NO:5, and a heavy chain CDR3 comprising the amino acid sequence of SEQ ID NO:6, wherein said human monoclonal antibody specifically binds to human IL-17RA, e.g. the monoclonal antibody is a human monoclonal antibody that specifically binds to human IL-17RA and inhibits the binding of IL-17A to said IL-17RA, wherein the composition is administered at a dose effective to reduce and maintain a NAPSI score 6 or less or mNAPSI of 3 or less on an affected nail.

Any of the preceding therapeutic agents, medicaments or compositions are used to treat a patient having about 10% to about 20% body surface area affected by psoriasis, such as plaque psoriasis, or about 20% to about 30% body surface area affected by psoriasis or about 30% to about 40% body surface area affected by psoriasis or about 40% to about 50% body surface area affected by psoriasis or about 50% to about 60% body surface area affected by psoriasis or about 60% to about 70% body surface area affected by psoriasis or about 70% to about 80% body surface area affected by psoriasis or about 80% to about 90% body surface area affected by psoriasis or about 90% to about 100% body surface area affected by psoriasis.

Another aspect of the invention provides for methods of treating nail or scalp psoriasis, comprising administering to a patient having less than 50% body surface area affected by psoriasis, such as plaque psoriasis, a composition comprising a antibody or fragment thereof selected from: an antibody, comprising a light chain variable domain comprising the amino acid sequence of SEQ ID NO:7 and a heavy chain variable domain comprising the amino acid sequence of SEQ ID NO:8; an antibody comprising the full length light chain of SEQ ID NO: 9 and a full length heavy chain of SEQ ID NO: 10 and an antibody, comprising a light chain CDR1 comprising the amino acid sequence of SEQ ID NO:1, a light chain CDR2 comprising the amino acid sequence of SEQ ID NO:2, a light chain CDR3 comprising the amino acid sequence of SEQ ID NO:3, a heavy chain CDR1 comprising the amino acid sequence of SEQ ID NO:4, a heavy chain CDR2 comprising the amino acid sequence of SEQ ID NO:5, and a heavy chain CDR3 comprising the amino acid sequence of SEQ ID NO:6, wherein said antibody specifically binds to human IL-17RA and inhibits the binding of IL-17A to said IL-17 receptor A. For example, the monoclonal antibody is a human monoclonal antibody that specifically binds to human IL-17RA and inhibits the binding of IL-17A to said IL-17RA. For example, in this method the patient has less than 50% body surface area affected by psoriasis, such as plaque psoriasis, or less than 40% body surface area affected by psoriasis or less than 30% body surface area affected by psoriasis, or less than 20% body surface area affected by psoriasis, or less than 10% body surface area affected by psoriasis.

Another aspect of the invention provides for use of an antibody or fragment thereof for the preparation of a medicament for the treatment of nail or scalp psoriasis, wherein the medicament is for administration to a patient having less than 50% body surface area affected by psoriasis, such as plaque psoriasis, wherein the antibody comprises a light chain variable domain comprising the amino acid sequence of SEQ ID NO:7 and a heavy chain variable domain comprising the amino acid sequence of SEQ ID NO:8; an antibody comprising the full length light chain of SEQ ID NO: 9 and a full length heavy chain of SEQ ID NO: 10 and an antibody, comprising a light chain CDR1 comprising the amino acid sequence of SEQ ID NO:1, a light chain CDR2 comprising the amino acid sequence of SEQ ID NO:2, a light chain CDR3 comprising the amino acid sequence of SEQ ID NO:3, a heavy chain CDR1 comprising the amino acid sequence of SEQ ID NO:4, a heavy chain CDR2 comprising the amino acid sequence of SEQ ID NO:5, and a heavy chain CDR3 comprising the amino acid sequence of SEQ ID NO:6, wherein said antibody specifically binds to human IL-17 receptor A and inhibits the binding of IL-17A to said IL-17RA. Such antibody may be a human monoclonal antibody that inhibits IL-17A binding to IL-17RA. For example, this medicament may be administered to patient having less than 50% body surface area affected by psoriasis, such as plaque psoriasis, or less than 40% body surface area affected by psoriasis or less than 30% body surface area affected by psoriasis, or less than 20% body surface area affected by psoriasis, or less than 10% body surface area affected by psoriasis or less than 7% body surface area affected by psoriasis or less than 5% body surface area affected by psoriasis or less than 2% body surface area affected by psoriasis.

Another aspect of the invention provides for methods of treating nail or scalp psoriasis, comprising administering to a patient having less than 10% body surface area affected by psoriasis, such as plaques psoriasis, a composition comprising a human antibody or fragment thereof selected from: an antibody, comprising a light chain variable domain comprising the amino acid sequence of SEQ ID NO:7 and a heavy chain variable domain comprising the amino acid sequence of SEQ ID NO:8; an antibody comprising the full length light chain of SEQ ID NO: 9 and a full length heavy chain of SEQ ID NO: 10 and an antibody, comprising a light chain CDR1 comprising the amino acid sequence of SEQ ID NO:1, a light chain CDR2 comprising the amino acid sequence of SEQ ID NO:2, a light chain CDR3 comprising the amino acid sequence of SEQ ID NO:3, a heavy chain CDR1 comprising the amino acid sequence of SEQ ID NO:4, a heavy chain CDR2 comprising the amino acid sequence of SEQ ID NO:5, and a heavy chain CDR3 comprising the amino acid sequence of SEQ ID NO:6, wherein said antibody specifically binds to human IL-17 receptor A. Such antibody may be a human monoclonal antibody that inhibits IL-17A binding to IL-17RA.

In one embodiment, the patient treated with the preceding method of the invention, or treated with a therapeutic agent, medicament or composition of the invention has 10% or less body surface area affected by moderate to severe nail or scalp psoriasis. Moderate to severe nail psoriasis includes patients with a NAPSI score of at least 6, or a mNAPSI score of at least 3 or at least 2 on one or more nails. For example, the patient with moderate to severe psoriasis has the presence of at least two of the following clinical features in at least one quarter of a nail: pitting, leukonychia, red spots in lunula, nail plate crumbling, oil drop (salmon patch) discoloration, onycholysis, nail bed hyperkeratosis, splinter hemorrhages or has at least one of these clinical features in more than one quarter of the one or more nails. Moderate to severe scalp psoriasis include patients having a PSSI score of at least 15 and/or a SSA score of at least 30%.

Another aspect of the invention provides for methods of treating nail psoriasis, comprising administering to a patient having a pretreatment NAPSI score of at least 6 or pretreatment mNAPSI score of at least 3 or at least 2 for at least one affected nail, a composition comprising an antibody or fragment thereof selected from: an antibody, comprising a light chain variable domain comprising the amino acid sequence of SEQ ID NO:7 and a heavy chain variable domain comprising the amino acid sequence of SEQ ID NO:8; an antibody comprising the full length light chain of SEQ ID NO: 9 and a full length heavy chain of SEQ ID NO: 10 and an antibody, comprising a light chain CDR1 comprising the amino acid sequence of SEQ ID NO:1, a light chain CDR2 comprising the amino acid sequence of SEQ ID NO:2, a light chain CDR3 comprising the amino acid sequence of SEQ ID NO:3, a heavy chain CDR1 comprising the amino acid sequence of SEQ ID NO:4, a heavy chain CDR2 comprising the amino acid sequence of SEQ ID NO:5, and a heavy chain CDR3 comprising the amino acid sequence of SEQ ID NO:6, wherein said human monoclonal antibody specifically binds to human IL-17RA and inhibits the binding of IL-17A to said IL-17RA, e.g. the monoclonal antibody is a human monoclonal antibody that specifically binds to human IL-17RA and inhibits the binding of IL-17A to said IL-17RA, and wherein the composition is administered at a dose and frequency effective to reduce the severity of the patient's nail or scalp psoriasis. In certain embodiments, the patient's score NAPSI score is reduced to 6 or lower. In another embodiment, the patient's mNAPSI score is reduced to 3 or lower on an affected nail. In another embodiment, the patient's NAPSI score is maintained at 6 or lower, or the patient's mNAPSI score is maintained at 3 or lower in an affected nail.

Another aspect of the invention provides for a use of an antibody or fragment thereof for the preparation of a medicament for the treatment of nail psoriasis, wherein the medicament is for administration to a patient having a pretreatment NAPSI score of at least 6 or pretreatment mNAPSI score of at least 3 or at least 2 for at least one affected nail, the medicament comprising an antibody or fragment thereof selected from: an antibody, comprising a light chain variable domain comprising the amino acid sequence of SEQ ID NO:7 and a heavy chain variable domain comprising the amino acid sequence of SEQ ID NO:8; an antibody comprising the full length light chain of SEQ ID NO: 9 and a full length heavy chain of SEQ ID NO: 10 and an antibody, comprising a light chain CDR1 comprising the amino acid sequence of SEQ ID NO:1, a light chain CDR2 comprising the amino acid sequence of SEQ ID NO:2, a light chain CDR3 comprising the amino acid sequence of SEQ ID NO:3, a heavy chain CDR1 comprising the amino acid sequence of SEQ ID NO:4, a heavy chain CDR2 comprising the amino acid sequence of SEQ ID NO:5, and a heavy chain CDR3 comprising the amino acid sequence of SEQ ID NO:6, wherein said h antibody specifically binds to human IL-17 receptor A, e.g. the monoclonal antibody is a human monoclonal antibody that specifically binds to human IL-17RA and inhibits the binding of IL-17A to said IL-17RA, and wherein the medicament is administered at a dose and frequency effective to reduce the severity of the patient's nail or scalp psoriasis. In certain embodiments, the patient's score NAPSI score is reduced to 6 or lower. In other embodiment, the patient's mNAPSI score is reduced to 3 or lower on an affected nail. In another embodiment, the patient's NAPSI score is maintained at 6 or lower, or the patient's mNAPSI score is maintained at 3 or lower on an affected nail.

Another aspect of the invention provides a composition for treatment of nail psoriasis, wherein the composition is for administration to a patient having a pretreatment NAPSI score of at least 6 or mNAPSI scores of at 3 or at least 2 for at least one affected nail, the composition comprising an human antibody or fragment thereof selected from: an antibody, comprising a light chain variable domain comprising the amino acid sequence of SEQ ID NO:7 and a heavy chain variable domain comprising the amino acid sequence of SEQ ID NO:8; an antibody comprising the full length light chain of SEQ ID NO: 9 and a full length heavy chain of SEQ ID NO: 10 and an antibody, comprising a light chain CDR1 comprising the amino acid sequence of SEQ ID NO:1, a light chain CDR2 comprising the amino acid sequence of SEQ ID NO:2, a light chain CDR3 comprising the amino acid sequence of SEQ ID NO:3, a heavy chain CDR1 comprising the amino acid sequence of SEQ ID NO:4, a heavy chain CDR2 comprising the amino acid sequence of SEQ ID NO:5, and a heavy chain CDR3 comprising the amino acid sequence of SEQ ID NO:6, wherein said human monoclonal antibody specifically binds to human IL-17 receptor A and inhibits the binding of IL-17A to said IL-17 receptor A, and wherein the composition is administered at a dose and frequency effective to reduce the severity of the patient's nail or scalp psoriasis. In certain embodiments, the patient's score NAPSI score is reduced to 6 or lower. In other embodiment, the patient's mNAPSI score is reduced to 3 or lower on an affected nail. In other embodiments, the patient's NAPSI score is maintained at 6 or lower, or the patient's mNAPSI score is maintained at 3 or lower.

In any of the preceding methods or any of the therapeutic agents, medicaments or compositions are administered to a patient suffering from psoriasis, such as plaque psoriasis and has about 9.75% or less body surface area affected by psoriasis or the patient has about 9.5% or less body surface area affected by psoriasis or the patient has about 9.25% or less body surface area affected by psoriasis, or the patient has about 9% or less body surface area affected by psoriasis, or the patient has about 8.5% or less body surface area affected by psoriasis, or the patient has about 8% or less body surface area affected by psoriasis or the patient has about 7.5% or less body surface area affected by psoriasis, or the patient has about 7% or less body surface area affected by psoriasis, or the patient has about 6.5% or less body surface area affected by psoriasis or the patient has about 6% or less body surface area affected by psoriasis, or the patient has about 5.5% or less body surface area affected by psoriasis, or the patient about 5% or less body surface area affected by psoriasis, or the patient has about 4.5% or less body surface area affected by psoriasis or the patient has about 4% or less body surface area affected by psoriasis or the patient has about 3.5% or less body surface area affected by psoriasis, or the patient has about 3% or less body surface area affected by psoriasis, or the patient has less than 2.75% body surface area affected by psoriasis, or the patient has about 2.5% or less body surface area affected by psoriasis, or the patient has about 2.5% or less body surface area affected by psoriasis, or the patient has about 2% or less body surface area affected by psoriasis, or the patient has about 1.75% or less body surface area affected by psoriasis, or the patient has about 1.5% or less body surface area affected by psoriasis, or the patient has about 1.25% or less body surface area affected by psoriasis, or the patient has about 1% or less body surface area affected by psoriasis, or the patient has about 0.9% body or less surface area affected by psoriasis, or the patient has about 0.8% or less body surface area affected by psoriasis, or the patient has about 0.7% or less body surface area affected by psoriasis, or the patient has about 0.6% or less body surface area affected by psoriasis, or the patient has about 0.5%% or less body surface area affected by psoriasis, or the patient has about 0.4% or less body surface area affected by psoriasis, or the patient has about 0.3% or less body surface area affected by psoriasis, or the patient has about 0.2% or less body surface area affected by psoriasis, or the patient has about 0.1% or less body surface area affected by psoriasis.

In any of the preceding methods or any of the therapeutic agents, medicaments or compositions are administered to a patient suffering from psoriasis, such as plaque psoriasis and has about 5% to about 9.9% body surface area affected by psoriasis or about 2.5% to about 9% body surface area affected by psoriasis or about 19% to about 9% body surface area affected by psoriasis or about 4% to about 8% body surface area affected by psoriasis or about 2% to about 8% body surface area affected by psoriasis or about 1% to about 8% body surface area affected by psoriasis or about 3% to about 7% body surface area affected by psoriasis or about 2% to about 7% body surface area affected by psoriasis or about 1% to about 7% body surface area affected by psoriasis or about 2% to about 6% body surface area affected by psoriasis or about 1% to about 6% body surface area affected by psoriasis or about 0.5% to about 6% body surface area affected by psoriasis or about 1% to about 5% body surface area affected by psoriasis or about 0.75% to about 5% body surface area affected by psoriasis or about 0.5% to about 5% body surface area affected by psoriasis or about 1% to about 4% body surface area affected by psoriasis or 0.75% to about 4% body surface area affected by psoriasis or about 0.5% to about 4% body surface area affected by psoriasis or about 0.25% to about 4% body surface area affected by psoriasis or 0.5% to about 2% body surface area affected by psoriasis or about 0.25% to about 2% body surface area affected by psoriasis or about 0.5% to about 1% body surface area affected by psoriasis.

Another aspect of the invention provides for methods of treating scalp psoriasis, comprising administering to a patient having a pretreatment PSSI score of at least 15 or least 10 or at least 5 or a pretreatment SSA score of at least 30%, or at least 20% or at least 10%, a composition comprising an antibody or fragment thereof selected from: an antibody, comprising a light chain variable domain comprising the amino acid sequence of SEQ ID NO:7 and a heavy chain variable domain comprising the amino acid sequence of SEQ ID NO:8; an antibody comprising the full length light chain of SEQ ID NO: 9 and a full length heavy chain of SEQ ID NO: 10, and an antibody, comprising a light chain CDR1 comprising the amino acid sequence of SEQ ID NO:1, a light chain CDR2 comprising the amino acid sequence of SEQ ID NO:2, a light chain CDR3 comprising the amino acid sequence of SEQ ID NO:3, a heavy chain CDR1 comprising the amino acid sequence of SEQ ID NO:4, a heavy chain CDR2 comprising the amino acid sequence of SEQ ID NO:5, and a heavy chain CDR3 comprising the amino acid sequence of SEQ ID NO:6, wherein said antibody specifically binds to human IL-17RA. The antibody or fragment thereof may be a human monoclonal antibody that and inhibits the binding of IL-17A to said IL-17 receptor A. In certain embodiments, the patient's PSSI score is reduced to 14 or lower. In another embodiment, the patient's PSSI score is maintained at 14 or lower. In another embodiment, the patients' SSA is reduced to 25% or less. In another embodiment, the patient's SSA is maintained at 25% or less.

Another aspect of the invention provides for an use of an antibody or fragment thereof for the preparation of a medicament for the treating scalp psoriasis, wherein the medicament is for administration to a patient having a pretreatment PSSI score of at least 15 or least 10 or at least 5 or a pretreatment SSA score of at least 30%, or at least 20% or at least 10%, and the antibody or fragment thereof is selected from: an antibody, comprising a light chain variable domain comprising the amino acid sequence of SEQ ID NO:7 and a heavy chain variable domain comprising the amino acid sequence of SEQ ID NO:8; an antibody comprising the full length light chain of SEQ ID NO: 9 and a full length heavy chain of SEQ ID NO: 10, and an antibody, comprising a light chain CDR1 comprising the amino acid sequence of SEQ ID NO:1, a light chain CDR2 comprising the amino acid sequence of SEQ ID NO:2, a light chain CDR3 comprising the amino acid sequence of SEQ ID NO:3, a heavy chain CDR1 comprising the amino acid sequence of SEQ ID NO:4, a heavy chain CDR2 comprising the amino acid sequence of SEQ ID NO:5, and a heavy chain CDR3 comprising the amino acid sequence of SEQ ID NO:6, wherein said antibody specifically binds to human IL-17RA. The antibody or fragment thereof may be a human monoclonal antibody that and inhibits the binding of IL-17A to said IL-17 receptor A. In certain embodiments, the patient's PSSI score is reduced to 14 or lower. In another embodiment, the patient's PSSI score is maintained at 14 or lower. In another embodiment, the patients' SSA is reduced to 25% or less. In another embodiment, the patient's SSA is maintained at 25% or less.

Another aspect of the invention provides compositions for use in treatment of scalp psoriasis, wherein the composition is for administration to a patient having a pretreatment PSSI score of at least 15 or least 10 or at least 5 or a pretreatment SSA score of at least 30%a, or at least 20% or at least 10%, and the antibody or fragment thereof is selected from: an antibody, comprising a light chain variable domain comprising the amino acid sequence of SEQ ID NO:7 and a heavy chain variable domain comprising the amino acid sequence of SEQ ID NO:8; an antibody comprising the full length light chain of SEQ ID NO: 9 and a full length heavy chain of SEQ ID NO: 10, and an antibody, comprising a light chain CDR1 comprising the amino acid sequence of SEQ ID NO:1, a light chain CDR2 comprising the amino acid sequence of SEQ ID NO:2, a light chain CDR3 comprising the amino acid sequence of SEQ ID NO:3, a heavy chain CDR1 comprising the amino acid sequence of SEQ ID NO:4, a heavy chain CDR2 comprising the amino acid sequence of SEQ ID NO:5, and a heavy chain CDR3 comprising the amino acid sequence of SEQ ID NO:6, wherein said antibody specifically binds to human IL-17RA. The antibody or fragment thereof may be a human monoclonal antibody that and inhibits the binding of IL-17A to said IL-17 receptor A. In certain embodiments, the patient's PSSI score is reduced to 14 or lower. In another embodiment, the patient's PSSI score is maintained at 14 or lower. In another embodiment, the patients' SSA is reduced to 25% or less.

For example, the preceding methods comprise administering a dose or any of the therapeutic agents, medicaments or compositions comprise a dose of the antibody or fragment thereof that is effective to reduce and maintain a PSSI score of 14 or less, or a PSSI score of 13 or less, a PSSI score of 12 or less, a PSSI score of 10 or less, a PSSI score of 9 or less, a PSSI score of 8 or less, a PSSI score of 7 or less, a PSSI score of 6 or less, a PSSI score of 5 or less, a PSSI score of 4 or less, a PSSI score of 3 or less, a PSSI score of 2 or less, a PSSI score of 1 or less. The dose of the antibody or fragment thereof of any of the preceding methods results in a PSSI score ranging from 12 to 14, or a PSSI score ranging from 10 to 14, or a PSSI score ranging from 8 to 14, or a PSSI score ranging from 6 to 14, or a PSSI score ranging from 4 to 14, or a PSSI score ranging from 2 to 14, or a PSSI score ranging from 10 to 12, or a PSSI score ranging from 8 to 12, or a PSSI score ranging from 6 to 12, or a PSSI score ranging from 4 to 12, or a PSSI score ranging from 2 to 12, or a PSSI score ranging from 8 to 10, or a PSSI score ranging from 6 to 10, or a PSSI score ranging from 4 to 10, or a PSSI score ranging from 2 to 10, or a PSSI score ranging from 6 to 8, or a PSSI score ranging from 4 to 8, or a PSSI score ranging from 2 to 8, or a PSSI score ranging from 4 to 6, or a PSSI score ranging from 2 to 6, or a PSSI score ranging from 2 to 4.

In another embodiment, the preceding methods comprise administering a dose or any of the, therapeutic agents, medicaments or compositions comprise a dose of the antibody or fragment thereof that is effective to reduce or maintain a SSA score of 25% or less, or a SSA score of 20% or less, or a SSA score of 15% or less, or a SSA score of 10% or less, or a SSA score of 5% or less, or a SSA score of 2% or less. The dose of the antibody or fragment thereof of any of the preceding methods results in a SSA score ranging from 20% to 30%, or a SSA score ranging from 15% to 30%, or a SSA score ranging from 10% to 30%, or a SSA score ranging from 20% to 25%, or a SSA score ranging from 15% to 25%, or a SSA score ranging from 10% to 25%, or a SSA score ranging from 5% to 25%, or a SSA score ranging from 15% to 20%, or a SSA score ranging from 10% to 20%, or a SSA score ranging from 5% to 20%, or a SSA score ranging from 12% to 15%, or a SSA score ranging from 10% to 15%, or a SSA score ranging from 5% to 15%, or a SSA score ranging from 2% to 15%, or a SSA score ranging from 7% to 10%, or a SSA score ranging from 5% to 10%, or a SSA score ranging from 2% to 10%, or a SSA score ranging from 5% to 7%, or a SSA score ranging from 2% to 7%, or a SSA score ranging from 2% to 5%.

In any of the preceding methods, the antigen binding protein such as an antibody or fragment thereof is administered to a patient in need thereof by subcutaneous injection such as subcutaneous autoinjection, intralesional injections, topical administration, or systemic administration via intravenous injection or infusion. The antigen binding protein may be administered alone or in combination with another treatment for nail or scalp psoriasis.

Any of the preceding thearpeutic agents, medicaments or composition are administered to a patient in need thereof by subcutaneous injection such as subcutaneous autoinjection, intralesional injections, topical administration, or systemic administration via intravenous injection or infusion. The therapeutic agent, medicament or composition may be administered alone or in combination with another treatment for nail or scalp psoriasis.

In any of the preceding methods, the antibody or fragment thereof is administered with a second treatment. In addition, any of the preceding therapeutic agents, medicaments and compositions may be administered with a second treatment. The second treatment is administered prior to, concurrent with, or subsequent to administration of said composition comprising said antibody or fragment thereof.

In one embodiment, the second treatment is a topical treatment, such as fluorouracil, dithranol, tazarotene, cyclosporine, calcineneurin inhibitors, triamcinolone, fluocinonide, topical steroids, vitamin D3, vitamin D3 analogs, betamethasone dipropionate, betamethasone valerate, calcipotriol, clobetasol, XAMIOL and DAIVOBET, coal tar, urea, corticosteroids, retinoids, anthralin, topical methatrexate, keratolytics, salicylic acid, tofacitinab, apremilast, topical JAK inhibitors and combinations thereof.

In an embodiment, the second treatment is a systemic treatment such as retinoids, acitretin cyclosporine, methotrexate, apremilast, tofacitinib, oral JAK inhibitors, oral PI3 kinase inhibitors, oral MAP kinase inhibitors, Fumaderm, fumarates, dimethyl fumarate, sulfasalazine, leflunomide, calcineurin inhibitors, azathioprine, thioguanine, hydroxyurea, hydroxychloroquine, sulfasalazine and antifungals and combinations thereof. The second treatment is also a biologic such as antagonist, e.g. antibody or chimeric protein, specific for TNF, IL-17, IL-12/23, or IL-23 such as infliximab, adalimumab, etanercept, alefacept, ustekinumab, ixekizumab, secukinumab, guselkumab, and combinations thereof. .

In another embodiment, the second treatment is triamcinolone acetonide photochemotherapy, laser therapy, Excimer laser, oral/topical psoralen with UVA (PUVA), pulsed dye laser, radiation therapy, superficial radiotherapy, electron beam therapy, Grenz ray therapy, ermatome shaving, aloe vera extract, narrow band U therapy, UV therapy and combinations thereof.

Any of the second treatments are combined such as a topical therapy combined with one or more systemic treatment, or a topical treatment combined with one or more of triamcinolone acetonide photochemotherapy, laser therapy, Excimer laser, oral/topical psorallen with UVA (PUVA), pulsed dye laser, radiation therapy, superficial radiotherapy, electron beam therapy, Grenz ray therapy, ermatome shaving, aloe vera extract, UV therapy or a systemic treatment combined with one or more of triamcinolone acetonide photochemotherapy, laser therapy, oral/topical psoralen with UVA (PUVA), pulsed dye laser, radiation therapy, superficial radiotherapy, electron beam therapy, Grenz ray therapy, ermatome shaving, aloe vera extract, narrow band U therapy and UV therapy and combinations thereofs.

In any of the methods, medicaments, compositions or the therapeutic agents of the invention, the antibody administered is selected from the group consisting of: a. a human antibody; b. a humanized antibody; c. a chimeric antibody; d. a monoclonal antibody; e. an antigen-binding antibody fragment; f. a single chain antibody; g. a diabody; h. a triabody; i. a tetrabody; j. a Fab fragment; k. a F(ab')2 fragment; 1. an IgD antibody; m. an IgE antibody; n. an IgM antibody; o. an IgG1 antibody; p. an IgG2 antibody; q. an IgG3 antibody; and r.an IgG4 antibody. In any of the methods of the invention, the antibody is a human IgG2 monoclonal antibody.

In any of the methods, medicaments, compositions or therapeutic agents of the invention, the composition administered is a pharmaceutical composition and that pharmaceutical composition further comprises a pharmaceutically acceptable diluent. In particular, the pharmaceutical composition comprises the following formulation that is an aqueous solution of a glutamic acid buffer and an IL-17RA antigen binding protein at a concentration of 100 to 150 mg/ml and wherein: a) said glutamic acid buffer comprises a glutamic acid concentration of 5-30 mM.+-.0.2 mM; b) said glutamic acid buffer comprises a pH of 4.5-5.2.+-.0.2; c) said formulation further comprises 2-4% proline (w/v) and 0.005-0.02% (w/v) polysorbate 20. This formulation has an osmolarity of 275 to 325 osm and has a viscosity of 5 to 7 cP at 25° C.

### BRIEF DESCRIPTION OF THE DRAWINGS

FIG. 1 provides the overall schema of the clinical study described in Example 1.

FIG. 2 provides the PSSI-75 measured in the study subjects every two weeks. The y-axis represents the percent of responders.

FIG. 3 provides the PSSI-100 measured in the study subjects every two weeks. The y-axis represents the percent of responders.

FIG. 4 provides the PSSI percent improvement from baseline. The y-axis represents the percent of responders.

FIG. 5 provides the NAPSI score by treatment group in the induction phase of the study in Example 1. The y-axis provides the NAPSI score as observed.

FIG. 6 provides the NAPSI score for non-rerandomized subjects by treatment group in the induction phase through week 52. The y-axis provides the NAPSI score as observed.

FIG. 7 provides the mean percent changes in NAPSI scores from baseline (panel A) and the mean percent changes in PSSI from baseline measured in the phase II study described in

Example 2. The horizontal axis shows week from initial administration of Brodalumab or placebo.

### DETAILED DESCRIPTION OF THE INVENTION

The section headings used herein are for organizational purposes only and are not to be construed as limiting the subject matter described.

Standard techniques are used for recombinant DNA, oligonucleotide synthesis, tissue culture and transformation, protein purification, etc. Enzymatic reactions and purification techniques are performed according to the manufacturer's specifications or as commonly accomplished in the art or as described herein. The following procedures and techniques are generally performed according to conventional methods well known in the art and as described in various general and more specific references that are cited and discussed throughout the specification. See, e.g., Sambrook et al., 2001, Molecular Cloning: A Laboratory Manual, 3rd ed., Cold Spring Harbor Laboratory Press, Cold Spring Harbor, N.Y., which is incorporated herein by reference for any purpose. Unless specific definitions are provided, the nomenclature used in connection with, and the laboratory procedures and techniques of, analytical chemistry, organic chemistry, and medicinal and pharmaceutical chemistry described herein are those well-known and commonly used in the art. Standard techniques are used for chemical synthesis, chemical analyses, pharmaceutical preparation, formulation, and delivery and treatment of patients.

### IL-17A, IL-17F, and IL-17RA

The biologic activities of IL-17A and IL-17F are dependent upon IL-17RA. “IL-17 receptor A” or “IL-17RA” (interchangeably used herein, as well as IL-17 receptor and IL-17R to refer to the same receptor) as used herein is meant the cell surface receptor and receptor complexes (such as but not limited to IL-17RA-IL-17RC complex and IL-17RA-IL-17RB). Without being bound to one particular theory, the different IL-17RA receptor complexes are known to bind one or more of the ligands: IL-17A , IL-17F, IL-17A/F and IL-17 C, and as a result initiate a signal transduction pathway within the cell. IL-17RA proteins also include variants. IL-17RA proteins also include fragments, such as the extracellular domain that don't have all or part of the transmembrane and/or the intracellular domain, as well as fragments of the extracellular domain. The cloning, characterization, and preparation of IL-17RA are described, for example, in U.S. Pat. No. 6,072,033, which is incorporated herein by reference in its entirety. The amino acid sequence of the human IL-17RA is shown in SEQ ID NO: 13. Soluble forms of huIL-17RA useful in the methods of the present invention include the extracellular domain or the mature form lacking the signal peptide or a fragment of the extracellular domain that retains the capacity to bind IL-17A and/or IL-17F, or a heteromeric version of IL-17A and/or IL-17F. Other forms of IL-17RA include muteins and variants that are at least between 70% and 99% homologous to the native IL-17RA of SEQ ID NO:13 and as described in U.S. Pat. No. 6,072,033, so long as the IL-17RA retains the capacity to bind IL-17A and/or IL-17F, or a heteromeric version of IL-17A and/or IL-17F. The term “IL-17RA” also includes post-translational modifications of the IL-17RA amino acid sequence. Post-translational modifications include, but is not limited to, N-and 0-linked glycosylation.

### IL-17RA Antigen Binding Proteins

The present invention also provides methods of treating scalp and nail psoriasis comprising administering an antigen binding proteins that specifically bind IL-17RA. The methods of the invention include administering an IL-17RA antigen binding protein described in U.S. Pat. No. 7,767,206, which is incorporated by reference herein in its entirety.

In a particular aspect, the invention provides for methods of treating scalp and nail psoriasis comprising administering an antibody comprising a light chain comprising the amino acid sequence of SEQ ID NO: 9 and a heavy chain comprising the amino acid sequence SEQ ID NO:10 or administering an antibody comprising a light chain encoded by the nucleotide sequence of SEQ ID NO: 11 and a heavy chain encoded by the nucleotide sequence of SEQ ID NO: 12. This antibody is described in detail in U.S Pat. No. 7,767,206, which is incorporated by reference herein in its entirety. This antibody is also referred to as brodalumab.

For example, the methods of the invention comprise administering an antibody that specifically binds IL-17RA and wherein said antibody comprises a light chain CDR1 having the amino acid sequence of SEQ ID NO: 1, the light chain CDR2 having the amino acid sequence of SEQ ID NO:2, the light chain CDR3 having the amino acid sequence of SEQ ID NO:3 and heavy chain CDR1 having the amino acid sequence of SEQ ID NO:4, the heavy chain CDR2 having the amino acid sequence of SEQ ID NO:5, and the heavy chain CDR3 having the amino acid sequence of SEQ ID NO:6; and fragments, derivatives, muteins, and variants thereof.

For example, embodiments of antigen binding proteins comprise peptides and/or polypeptides (that optionally include post-translational modifications) that specifically bind IL-17RA to a subject in need. Embodiments of antigen binding proteins comprise antibodies and fragments thereof, as variously defined herein, that specifically bind IL-17RA. Aspects of the invention include antibodies that specifically bind to IL-17RA and have antagonistic activities. The antagonistic activity in the present invention may be any activity as long as the activity inhibits a biological response of IL-17RA irrespective of whether the binding between IL-17RA and a ligand of the receptor is inhibited or not. Specific examples of the biological response include proliferation, infiltration and migration of an IL-17RA expressing cell, a cytokine production from an IL-17RA expressing cell and the like. Aspects of the invention include antibodies that specifically bind to human IL-17RA and inhibit IL-17A and/or IL-17F from binding and activating IL-17RA, or a heteromeric complex of IL-17RA and IL-17RC. Throughout the specification, when reference is made to inhibiting IL-17A and/or IL-17F, it is understood that this also includes inhibiting heteromers of IL-17A and IL-17F. Aspects of the invention include antibodies that specifically bind to human IL-17RA and partially or fully inhibit IL-17RA from forming either a homomeric or heteromeric functional receptor complex, such as, but not limited to, an IL-17RA-IL-17RC complex. Aspects of the invention include antibodies that specifically bind to human IL-17RA and partially or fully inhibit IL-17RA from forming either a homomeric or heteromeric functional receptor complex, such as, but not limited to IL-17RA/IL-17RC complex and do not necessarily inhibit IL-17A and/or IL-17F or an IL-17A/IL-17F heteromer from binding to IL-17RA or a IL-17RA heteromeric receptor complex.

The antigen binding proteins of the invention specifically bind to IL-17RA. “Specifically binds” as used herein means that the antigen binding protein preferentially binds IL-17RA over other proteins. In some embodiments “specifically binds” means that the IL-17RA antigen binding proteins have a higher affinity for IL-17RA than for other proteins. For example, the equilibrium dissociation constant is <10-7 to 10-11 M, or <10-8 to <10-10 M, or <10-9 to <10-10 M.

It is understood that when reference is made to the various embodiments of the IL-17RA antibodies described herein, that it also encompasses IL-17RA-binding fragments thereof. An IL-17RA-binding fragment comprises any of the antibody fragments or domains described herein that retains the ability to specifically bind to IL-17RA. Said IL-17RA-binding fragments are in any of the scaffolds described herein. Said IL-17RA-binding fragments also have the capacity to inhibit activation of the IL-17RA, as described throughout the specification.

In a further variation, the antigen binding protein comprises A) a heavy chain amino acid sequence that comprises a H-CDR1, a H-CDR2, and a H-CDR3 of any of SEQ ID NO:4-6, and B) a light chain amino acid sequence that comprises a L-CDR1, a L-CDR2, and a L-CDR3 of any of SEQ ID NO:1-3. In another variation, the antigen binding protein comprises an amino acid sequence that is of at least 80%, 81%, 82%, 83%, 84%, 85%, 86%, 87%, 88%, 89%, 90%, 91%, 92%, 93%, 94%, 95%, 96%, 97%, 98%, or 99% identical to a heavy chain amino acid sequence selected from the group consisting of SEQ ID NO:4-6 or a light chain amino acid sequence selected from the group consisting of SEQ ID NO:1-3.

In another embodiment, the invention provides an antigen binding protein that specifically binds IL-17RA, wherein said antigen binding protein comprises a light chain CDR1, CDR2, CDR3 and a heavy chain CDR1, CDR2, and CDR3 that differs by no more than a total of one, two, three, four, five, or six amino acid additions, substitutions, and/or deletions from the following CDR sequences:; light chain CDR1 (SEQ ID NO:1), CDR2 (SEQ ID NO:2), CDR3 (SEQ ID NO:3) and heavy chain CDR1 (SEQ ID NO:4), CDR2 (SEQ ID NO:5), CDR3 (SEQ ID NO:5) of antibody AM-14; and fragments, derivatives, muteins, and variants thereof.

In another embodiment, the light chain variable domain comprises a sequence of amino acids that is encoded by a polynucleotide that hybridizes under moderately stringent conditions to a complement of a light chain polynucleotide sequence of SEQ ID NO: 11

In another embodiment, the heavy chain variable domain comprises a sequence of amino acids that is encoded by a polynucleotide that hybridizes under moderately stringent or stringent conditions to a complement of a heavy chain polynucleotide sequence of SEQ ID NO: 12.

Accordingly, in various embodiments, the antigen binding proteins of the invention comprise the scaffolds of traditional antibodies, including human and monoclonal antibodies, bispecific antibodies, diabodies, minibodies, domain antibodies, synthetic antibodies (sometimes referred to herein as “antibody mimetics”), chimeric antibodies, antibody fusions (sometimes referred to as “antibody conjugates”), and fragments of each, respectively. The above described CDRs and combinations of CDRs are grafted into any of the following scaffolds.

As used herein, the term “antibody” refers to the various forms of monomeric or multimeric proteins comprising one or more polypeptide chains that specifically binds to an antigen, as variously described herein. In certain embodiments, antibodies are produced by recombinant DNA techniques. In additional embodiments, antibodies are produced by enzymatic or chemical cleavage of naturally occurring antibodies. In another aspect, the antibody is selected from the group consisting of: a) a human antibody; b) a humanized antibody; c) a chimeric antibody; d) a monoclonal antibody; e) a polyclonal antibody; f) a recombinant antibody; g) an antigen-binding antibody fragment; h) a single chain antibody; i) a diabody; j) a triabody; k) a tetrabody; 1) a Fab fragment; m) a F(ab')2 fragment; n) an IgD antibody; o) an IgE antibody; p) an IgM antibody; q) an IgA antibody; r) an IgG1 antibody; s) an IgG2 antibody; t) an IgG3 antibody; and u) an IgG4 antibody.

“Humanized antibodies” generally refer to non-human antibodies that have had the variable-domain framework regions swapped for sequences found in human antibodies. Generally, in a humanized antibody, the entire antibody, except the CDRs, is encoded by a polynucleotide of human origin or is identical to such an antibody except within its CDRs. The CDRs, some or all of which are encoded by nucleic acids originating in a non-human organism, are grafted into the beta-sheet framework of a human antibody variable region to create an antibody, the specificity of which is determined by the engrafted CDRs. The creation of such antibodies is described in, e.g., WO 92/11018, Jones, 1986, Nature 321:522-525, Verhoeyen et al., 1988, Science 239:1534-1536. Humanized antibodies can also be generated using mice with a genetically engineered immune system. Roque et al., 2004, Biotechnol. Prog. 20:639-654. In the present invention, the identified CDRs are human, and thus both humanized and chimeric antibodies in this context include some non-human CDRs; for example, humanized antibodies are generated that comprise the CDRH3 and CDRL3 regions, with one or more of the other CDR regions being of a different special origin.

In one embodiment, the IL-17RA antigen binding protein is a multispecific antibody, and notably a bispecific antibody, also sometimes referred to as “diabodies”. These are antibodies that bind to two (or more) different antigens. Diabodies can be manufactured in a variety of ways known in the art (Holliger and Winter, 1993, Current Opinion Biotechnol. 4:446-449), e.g., prepared chemically or from hybrid hybridomas.

In one embodiment, the IL-17RA antigen binding protein is a minibody. Minibodies are minimized antibody-like proteins comprising a scFv joined to a CH3 domain. Hu et al., 1996, Cancer Res. 56:3055-3061.

In one embodiment, the IL-17RA antigen binding protein is an antibody fragment, that is a fragment of any of the antibodies outlined herein that retain binding specificity to IL-17RA. In various embodiments, the antibody binding proteins comprise, but are not limited to, a F(ab), F(ab'), F(ab')2, Fv, or a single chain Fv fragments. At a minimum, an antibody, as meant herein, comprises a polypeptide binds specifically to IL-17RA comprising all or part of a light or heavy chain variable region, such as one or more CDRs.

Further examples of IL-17RA-binding antibody fragments include, but are not limited to, (i) the Fab fragment consisting of VL, VH, CL and CH1 domains, (ii) the Fd fragment consisting of the VH and CH1 domains, (iii) the Fv fragment consisting of the VL and VH domains of a single antibody; (iv) the dAb fragment (Ward et al., 1989, Nature 341:544-546) which consists of a single variable, (v) isolated CDR regions, (vi) F(ab')2 fragments, a bivalent fragment comprising two linked Fab fragments (vii) single chain Fv molecules (scFv), wherein a VH domain and a VL domain are linked by a peptide linker which allows the two domains to associate to form an antigen binding site (Bird et al., 1988, Science 242:423-426, Huston et al., 1988, Proc. Natl. Acad. Sci. U.S.A. 85:5879-5883), (viii) bispecific single chain Fv dimers (PCT/US92/09965) and (ix) “diabodies” or “triabodies”, multivalent or multispecific fragments constructed by gene fusion (Tomlinson et. al., 2000, Methods Enzymol. 326:461-479; W094/13804; Holliger et al., 1993, Proc. Natl. Acad. Sci. U.S.A. 90:6444-6448). The antibody fragments are modified. For example, the molecules are stabilized by the incorporation of disulphide bridges linking the VH and VL domains (Reiter et al., 1996, Nature Biotech. 14:1239-1245). Aspects of the invention include embodiments wherein the non-CDR components of these fragments are human sequences.

In one embodiment, the IL-17RA antigen binding protein is a fully human antibody. In this embodiment, as outlined above, specific structures comprise complete heavy and light chains depicted comprising the CDR regions. Additional embodiments utilize one or more of the CDRs of the invention, with the other CDRs, framework regions, J and D regions, constant regions, etc., coming from other human antibodies. For example, the CDRs of the invention can replace the CDRs of any number of human antibodies, particularly commercially relevant antibodies

Single chain antibodies are formed by linking heavy and light chain variable domain (Fv region) fragments via an amino acid bridge (short peptide linker), resulting in a single polypeptide chain. Such single-chain Fvs (scFvs) have been prepared by fusing DNA encoding a peptide linker between DNAs encoding the two variable domain polypeptides (VL and VH). The resulting polypeptides can fold back on themselves to form antigen-binding monomers, or they can form multimers (e.g., dimers, trimers, or tetramers), depending on the length of a flexible linker between the two variable domains (Kortt et al., 1997, Prot. Eng. 10:423; Kortt et al., 2001, Biomol. Eng. 18:95-108). By combining different VL and VH-comprising polypeptides, one can form multimeric scFvs that bind to different epitopes (Kriangkum et al., 2001, Biomol. Eng. 18:31-40). Techniques developed for the production of single chain antibodies include those described in U.S. Pat. No. 4,946,778; Bird, 1988, Science 242:423; Huston et al., 1988, Proc. Natl. Acad. Sci. USA 85:5879; Ward et al., 1989, Nature 334:544, de Graaf et al., 2002, Methods Mol Biol. 178:379-87.

By “protein,” as used herein, is meant at least two covalently attached amino acids, which includes proteins, polypeptides, oligopeptides and peptides. In some embodiments, the two or more covalently attached amino acids are attached by a peptide bond. The protein is made up of naturally occurring amino acids and peptide bonds, for example when the protein is made recombinantly using expression systems and host cells, as outlined below. Alternatively, the protein includes synthetic amino acids (e.g., homophenylalanine, citrulline, ornithine, and norleucine), or peptidomimetic structures, i.e., “peptide or protein analogs”, such as peptoids (see, Simon et al., 1992, Proc. Natl. Acad. Sci. U.S.A. 89:9367, incorporated by reference herein), which can be resistant to proteases or other physiological and/or storage conditions. Such synthetic amino acids is incorporated in particular when the antigen binding protein is synthesized in vitro by conventional methods well known in the art. In addition, any combination of peptidomimetic, synthetic and naturally occurring residues/structures can be used. “Amino acid” also includes imino acid residues such as proline and hydroxyproline. The amino acid “R group” or “side chain” is either the (L)- or the (S)-configuration. In a specific embodiment, the amino acids are in the (L)- or (S)-configuration.

In certain aspects, the invention provides recombinant antigen binding proteins that bind an IL-17RA, in some embodiments a recombinant human IL-17RA or portion thereof. In this context, a “recombinant protein” is a protein made using recombinant techniques using any techniques and methods known in the art, i.e., through the expression of a recombinant nucleic acid as described herein. Methods and techniques for the production of recombinant proteins are well known in the art. Embodiments of the invention include recombinant antigen binding proteins that bind wild-type IL-17RA and variants thereof.

### Use of IL-17RA Antigen Binding Proteins for Diagnostic and Therapeutic Purposes

The IL-17RA antigen binding proteins of the present invention is used for the prevention or treatment of diseases or conditions associated with the IL-17A and/or IL-17F activity. For example, the IL-17RA antigen binding domains of the present are used for the prevention and treatment of scalp and/or nail psoriasis.

Antigen binding proteins of the invention, e.g. antibodies and fragments thereof, that specifically bind to IL-17RA may be used in treatment of scalp or nail psoriasis in a patient in need thereof. The antigen binding proteins of the invention, e.g. antibodies and fragmentes thereof, that specifically bind to IL-17A may be administered systemically via intravenous injection or infusion, subcutaneous injection such as subcutaneous autoinjection, intralesional injections or topical administration. The antigen binding protein may be administered alone or in combination with another treatment for nail or scalp psoriasis.

All aspects of the IL-17RA antigen binding proteins described throughout this specification may be used in the preparation of a medicament for the treatment of scalp or nail psoriasis described herein. In addition, the IL-17RA antigen binding protein of the invention are used to inhibit IL-17RA from forming a complex with its ligand, e.g., IL-17A and/or IL-17F or any other IL-17 ligand family member that binds IL-17RA or a heterologous complex comprising IL-17RA and IL-17RC, thereby modulating the biological activity of IL-17RA in a cell or tissue. Antigen binding proteins that bind to IL-17RA thus may modulate and/or inhibit interaction with other binding compounds and as such may have therapeutic use in ameliorating scalp or nail psoriasis. In specific embodiments, IL-17RA antigen binding proteins may inhibit IL-17A and/or IL-17F and/or IL-17A/F from binding IL-17RA, which may result in disruption of the IL-17RA-induced signal transduction cascade.

Topical treatments for nail psoriasis include fluorouracil, dithranol, anthralin, tazarotene, cyclosporine, calcineeneurin inhibitors triamcinolone, fluocinonide, topical steroids, corticosteroids, vitamin D3, vitamin D3 analogs such as betamethasone dipropionate, betamethasone valerate, calcipotriol (e.g DAIVOBET) clobetasol, combination therapies such as XAMIOL (betamethasome dipropionate and calcipotriol gel) and combinations thereof.

Systemic treatments for nail psoriasis include a systemic treatment such as retinoids, acitretin cyclosporine, antifungals, methotrexate and biologic therapies such as such as antagonists, e.g. antibody or chimeric protein, specific for TNF IL-17 IL-12/23 or IL-23 such as infliximab, adalimumab, etanercept, alefacept and ustekinumab and combinations thereof.

Other types of treatment for nail psoriasis include triamcinolone acetonide photochemotherapy, narrow band phototherapy, photochemotherapy with UVA, photosensitizer psoralan with UVA (PUVA), laser therapy, pulsed dye laser, radiation therapy, superficial radiotherapy, electron beam therapy and Grenz ray therapy, and combinations thereof.

Topical treatments for scalp psoriasis include corticosteroids such as hydrocortisone, clobetasone, triamcinolone, betamethasome verate, betamethasone dipropionate, desoximethasone, salicylic acid, coal tar, zinc pyrithion, antifungals, dithranol, antimycotics, vitamin D3, vitamin D3 analogs, urea, retinoids, anthralin, topical methotrexate and keratolytics and combinations thereof.

Systemic treatment for scalp psoriasis include methrotrexate, cyclosporine, acritretin, and biologic therapies such as such as antagonists, e.g. antibody or chimeric protein, specific for TNF IL-17 IL-12/23 or IL-23 such as infliximab, adalimumab, etanercept, and alefacept and combinations thereof.

Other types of treatment for scalp psoriasis include photochemical therapy, photosensitizer psoralan (PUVA) psoralelectron beam therapy and Grenz ray therapy, dermatome shaving, aloe vera extract and UV therapy and combinations thereof.

Treatment of scalp or nail psoriasis includes the use of first line drugs for topical treatments or other drugs for control of pain and inflammation in combination (pretreatment, post-treatment, or concurrent treatment) with treatment with one or more of the antigen binding proteins provided herein. These drugs are classified as non-steroidal, anti-inflammatory drugs (NSAIDs). Secondary treatments include corticosteroids, slow acting antirheumatic drugs (SAARDs), or disease modifying (DM) drugs. Information regarding the following compounds can be found in The Merck Manual of Diagnosis and Therapy, Sixteenth Edition, Merck, Sharp & Dohme Research Laboratories, Merck & Co., Rahway, N.J. (1992) and in Pharmaprojects, PJB Publications Ltd.

The antigen binding proteins described herein are used in combination (pre-treatment, post-treatment, or concurrent treatment) with any of one or more TNF inhibitors for the treatment or prevention of scalp or nail psoriasis, such as but not limited to, all forms of soluble TNF receptors including Etanercept (such as ENBREL®), as well as all forms of monomeric or multimeric p75 and/or p55 TNF receptor molecules and fragments thereof; anti-human TNF antibodies, such as but not limited to, Infliximab (such as REMICADE®), and D2E7 (such as HUMIRA®), and the like. Such TNF inhibitors include compounds and proteins which block in vivo synthesis or extracellular release of TNF. In a specific embodiment, the present invention is directed to the use of an IL-17RA antigen binding protein in combination (pre-treatment, post-treatment, or concurrent treatment) with any of one or more of the following TNF inhibitors: TNF binding proteins (soluble TNF receptor type-I and soluble TNF receptor type-II (“sTNFRs”), as defined herein), anti-TNF antibodies, granulocyte colony stimulating factor; thalidomide; BN 50730; tenidap; E 5531; tiapafant PCA 4248; nimesulide; PANAVIR® (Probucol); rolipram; RP 73401; peptide T; MDL 201,449A; (1R,35)-Cis-1-[9-(2,6-diaminopurinyl)]-3-hydroxy-4-cyclopentene hydrochloride; (1R,3R)-trans-1-(9-(2,6-diamino)purine]-3-acetoxycyclopentane; (1R,3R)-trans-1-[9-adenyl)-3-azidocyclopentane hydrochloride and (1R,3R)-trans-1-(6-hydroxy-purin-9-yl)-3-azidocyclo-pentane. TNF binding proteins are disclosed in the art (EP 308 378, EP 422 339, GB 2 218 101, EP 393 438, WO 90/13575, EP 398 327, EP 412 486, WO 91/03553, EP 418 014, JP 127,800/1991, EP 433 900, U.S. Pat. No. 5,136,021, GB 2 246 569, EP 464 533, WO 92/01002, WO 92/13095, WO 92/16221, EP 512 528, EP 526 905, WO 93/07863, EP 568 928, WO 93/21946, WO 93/19777, EP 417 563, WO 94/06476, and PCT International Application No. PCT/US97/12244).

For example, EP 393 438 and EP 422 339 teach the amino acid and nucleic acid sequences of a soluble TNF receptor type I (also known as “sTNFR-I” or “30kDa TNF inhibitor”) and a soluble TNF receptor type II (also known as “sTNFR-II” or “40kDa TNF inhibitor”), collectively termed “sTNFRs”, as well as modified forms thereof (e.g., fragments, functional derivatives and variants). EP 393 438 and EP 422 339 also disclose methods for isolating the genes responsible for coding the inhibitors, cloning the gene in suitable vectors and cell types and expressing the gene to produce the inhibitors. Additionally, polyvalent forms (i.e., molecules comprising more than one active moiety) of sTNFR-I and sTNFR-II have also been disclosed. In one embodiment, the polyvalent form is constructed by chemically coupling at least one TNF inhibitor and another moiety with any clinically acceptable linker, for example polyethylene glycol (WO 92/16221 and WO 95/34326), by a peptide linker (Neve et al. (1996), Cytokine, 8(5):365-370, by chemically coupling to biotin and then binding to avidin (WO 91/03553) and, finally, by combining chimeric antibody molecules (U.S. Pat. No. 5,116,964, WO 89/09622, WO 91/16437 and EP 315062.

The antigen binding proteins described herein are used in combination with all forms of CD28 inhibitors, such as but not limited to, abatacept (for example ORENCIA®).

The antigen binding proteins described herein are used in combination with all forms of IL-6 and/or IL-6 receptor inhibitors, such as but not limited to, tocilizumab (for example ACTEMRA®).

The antigen binding proteins described herein are used in combination with all forms of IL-23 and/or IL-12 such as ustekinumab (STELARA) and guselkumab.

The antigen binding protein described herein are used in combination with other IL-17RA inhibitors, such as secukinumab and ixekizumab.

The antigen binding proteins described herein are used in combination with small molecules that bind and/or inhibit IL-17RA or IL-17 activity or small molecules that bind and/or inhibit the activity of other pro-inflammatory cytokines. Examples of these small molecules include synthetic small molecule macrocycle antagonists of human IL-17A as those described in Livingston et al. “Identification and Characterization of Synthetic Small Molecule Macrocycle Antagonists of Human IL17A” ACR Annual Meeting, November 9-14, 2012, which is incorporated by reference herein in its entirety.

In a specific embodiment, the present invention is directed to the use of an antigen binding protein and any of one or more NSAIDs for the treatment of the diseases and disorders recited herein. NSAIDs owe their anti-inflammatory action, at least in part, to the inhibition of prostaglandin synthesis (Goodman and Gilman in “The Pharmacological Basis of Therapeutics,” MacMillan 7th Edition (1985)). NSAIDs can be characterized into at least nine groups: (1) salicylic acid derivatives; (2) propionic acid derivatives; (3) acetic acid derivatives; (4) fenamic acid derivatives; (5) carboxylic acid derivatives; (6) butyric acid derivatives; (7) oxicams; (8) pyrazoles and (9) pyrazolones.

In another specific embodiment, the present invention is directed to the use of an antigen binding protein in combination (pretreatment, post-treatment, or concurrent treatment) with any of one or more salicylic acid derivatives, prodrug esters or pharmaceutically acceptable salts thereof. Such salicylic acid derivatives, prodrug esters and pharmaceutically acceptable salts thereof comprise: acetaminosalol, aloxiprin, aspirin, benorylate, bromosaligenin, calcium acetylsalicylate, choline magnesium trisalicylate, magnesium salicylate, choline salicylate, diflusinal, etersalate, fendosal, gentisic acid, glycol salicylate, imidazole salicylate, lysine acetylsalicylate, mesalamine, morpholine salicylate, 1-naphthyl salicylate, olsalazine, parsalmide, phenyl acetylsalicylate, phenyl salicylate, salacetamide, salicylamide O-acetic acid, salsalate, sodium salicylate and sulfasalazine. Structurally related salicylic acid derivatives having similar analgesic and anti-inflammatory properties are also intended to be encompassed by this group.

In still another specific embodiment, the present invention is directed to the use of an antigen binding protein in combination (pretreatment, post-treatment or concurrent treatment) with any of one or more corticosteroids, prodrug esters or pharmaceutically acceptable salts thereof for the treatment of the diseases and disorders recited herein, including acute and chronic inflammation such as rheumatic diseases, graft versus host disease and multiple sclerosis. Corticosteroids, prodrug esters and pharmaceutically acceptable salts thereof include hydrocortisone and compounds which are derived from hydrocortisone, such as 21-acetoxypregnenolone, alclomerasone, algestone, amcinonide, beclomethasone, betamethasone, betamethasone valerate, budesonide, chloroprednisone, clobetasol, clobetasol propionate, clobetasone, clobetasone butyrate, clocortolone, cloprednol, corticosterone, cortisone, cortivazol, deflazacon, desonide, desoximerasone, dexamethasone, diflorasone, diflucortolone, difluprednate, enoxolone, fluazacort, flucloronide, flumethasone, flumethasone pivalate, flucinolone acetonide, flunisolide, fluocinonide, fluorocinolone acetonide, fluocortin butyl, fluocortolone, fluocortolone hexanoate, diflucortolone valerate, fluorometholone, fluperolone acetate, fluprednidene acetate, fluprednisolone, flurandenolide, formocortal, halcinonide, halometasone, halopredone acetate, hydro-cortamate, hydrocortisone, hydrocortisone acetate, hydro-cortisone butyrate, hydrocortisone phosphate, hydrocortisone 21-sodium succinate, hydrocortisone tebutate, mazipredone, medrysone, meprednisone, methylprednisolone, mometasone furoate, paramethasone, prednicarbate, prednisolone, prednisolone 21-diedryaminoacetate, prednisolone sodium phosphate, prednisolone sodium succinate, prednisolone sodium 21-m-sulfobenzoate, prednisolone sodium 21-stearoglycolate, prednisolone tebutate, prednisolone 21-trimethylacetate, prednisone, prednival, prednylidene, prednylidene 21-diethylaminoacetate, tixocortol, triamcinolone, triamcinolone acetonide, triamcinolone benetonide and triamcinolone hexacetonide. Structurally related corticosteroids having similar analgesic and anti-inflammatory properties are also intended to be encompassed by this group.

The antigen binding proteins are used to reduce IL-17RA activity, comprising administering an antigen binding protein. The present invention is also directed to methods of inhibiting binding and/or signaling of IL-17A and/or IL-17F to IL-17RA comprising providing the antigen binding protein of the invention to IL-17RA. In certain embodiments, the antigen binding protein inhibits binding and/or signaling of IL-17A and IL-17F to IL-17RA. In additional embodiments, the antigen binding protein inhibits binding and/or signaling of IL-17A but not IL-17F to IL-17RA. In other embodiments, the antigen binding protein inhibits binding and/or signaling of IL-17F and not IL-17A to IL-17RA. The antigen binding proteins are used in treating the consequences, symptoms, and/or the pathology associated with IL-17RA activity, comprising administering an antigen binding protein. The antigen binding proteins are used to inhibit the production of one or more of an inflammatory cytokine, chemokine, matrix metalloproteinase, or other molecule associated with IL-17RA activation, comprising administering an antigen binding protein. The antigen binding proteins are used in methods of inhibiting production of molecules such as but is not limited to: IL-6, IL-8, CXCL1, CXCL2, GM-CSF, G-CSF, M-CSF, IL-113, TNFa, RANK-L, LIF, PGE2, IL-12, MMPs (such as but not limited to MMP3 and MMP9), GROa, NO, and/or C-telopeptide and the like, comprising administering an antigen binding protein. The antigen binding proteins inhibit proinflammatory and proautoimmune immune responses and are used to treat diseases associated with activity of the IL-17A and/or IL-17F/IL-17RA pathway.

### Methods of Treatment: Pharmaceutical Formulations, Routes of Administration

In some embodiments, the invention provides pharmaceutical compositions comprising a therapeutically effective amount of one or a plurality of the antigen binding proteins of the invention together with a pharmaceutically acceptable diluent, carrier, solubilizer, emulsifier, preservative, and/or adjuvant. In addition, the invention provides methods of treating a patient by administering such pharmaceutical composition. The term “patient” includes human and animal subjects.

The methods of the invention comprise administering a composition comprising an IL-17RA antigen binding protein formulation including any formulation described in U.S. Pat. No. 7,767,206, which is incorporated by reference herein in its entirety.

In another embodiment, the IL-17 antigen binding protein formulation used in the methods of the invention is one of the formulation described in U.S Patent Publication No. US2013/0022621, which is incorporated herein by reference in its entirety. For example, a pharmaceutical formulation, comprising an aqueous solution of a glutamic acid buffer and an antibody comprising a heavy chain CDR1 comprising SEQ ID NO:4, a heavy chain CDR2 comprising SEQ ID NO:5, a heavy chain CDR3 comprising SEQ ID NO:6, a light chain CDR1 comprising SEQ ID NO:1, a light chain CDR2 comprising SEQ ID NO:2, and a light chain CDR3 comprising SEQ ID NO:3, wherein said antibody, or fragment thereof, specifically binds human IL-17 receptor A, and wherein: a) said glutamic acid buffer comprises a glutamic acid concentration of 5-30 mM.+-.0.2 mM; b) said glutamic acid buffer comprises a pH of 4.5-5.2.+−0.2; c) said formulation further comprises 2-4% proline (w/v) and 0.005-0.02% (w/v) polysorbate 20; and d) said antibody is at a concentration of 100 to 150 mg/ml. This formulation has an osmolarity of 275 to 325 osm and has a viscosity of 5 to 7 cP at 25oC.

In one embodiment, the IL-17RA antigen binding protein composition comprises an aqueous solution of a glutamic acid buffer and an antibody or a fragment thereof comprising a heavy chain CDR1 comprising SEQ ID NO:4, a heavy chain CDR2 comprising SEQ ID NO:5, a heavy chain CDR3 comprising SEQ ID NO:6, a light chain CDR1 comprising SEQ ID NO:1, a light chain CDR2 comprising SEQ ID NO:3, and a light chain CDR3 comprising SEQ ID NO:3, wherein said antibody, or fragment thereof, specifically binds human IL-17 receptor A, and wherein: said formulation comprises a glutamic acid concentration of 10±0.2 mM; said formulation has a pH of 4.5-5.2±0.2; said formulation further comprises 3±0.2% proline (w/v) and 0.01±0.002% (w/v) polysorbate 20; said antibody is at a concentration of about 140±5% mg/ml; and said formulation has a viscosity of 5 to 7 cP at 25 degrees C. This formulation has an osmolarity of 275 to 325 osm.

In one embodiment, the IL-17RA antigen binding domain composition comprise an aqueous solution of a glutamic acid buffer and an antibody or a fragment thereof comprising a heavy chain CDR1 comprising SEQ ID NO:4, a heavy chain CDR2 comprising SEQ ID NO:5, a heavy chain CDR3 comprising SEQ ID NO:6, a light chain CDR1 comprising SEQ ID NO:1, a light chain CDR2 comprising SEQ ID NO:2, and a light chain CDR3 comprising SEQ ID NO:3, wherein said antibody, or fragment thereof, specifically binds human IL-17 receptor A, and wherein: said formulation has a glutamic acid concentration of 10±0.2 mM; said formulation has a pH of 4.8±0.2; said formulation further comprises 3±0.2% proline (w/v) and 0.01±0.002% (w/v) polysorbate 20; said antibody is at a concentration of about 140±5% mg/ml; and said formulation has a viscosity of 5 to 7 cP at 25 degrees C. This formulation has an osmolarity of 275 to 325 osm.

In another embodiment, the IL-17RA antigen binding protein composition comprises an aqueous solution of a glutamic acid buffer and an antibody or a fragment thereof comprising a heavy chain CDR1 comprising SEQ ID NO:4, a heavy chain CDR2 comprising SEQ ID NO:5, a heavy chain CDR3 comprising SEQ ID NO:6, a light chain CDR1 comprising SEQ ID NO:1, a light chain CDR2 comprising SEQ ID NO:3, and a light chain CDR3 comprising SEQ ID NO:3, wherein said antibody, or fragment thereof, specifically binds human IL-17 receptor A, and wherein: said formulation comprises 30 mM glutamic acid; said formulation has a pH of 4.8±0.2; said formulation further comprises 2.4±0.2% proline (w/v) and 0.01±0.002% (w/v) polysorbate 20; said antibody is at a concentration of about 140±5% mg/ml; and said formulation has a viscosity of 5 to 7 cP at 25 degrees C. This formulation has an osmolarity of 275 to 325 osm.

Preferably, acceptable formulation materials are nontoxic to recipients at the dosages and concentrations employed. In specific embodiments, pharmaceutical compositions comprising a therapeutically effective amount of IL-17RA antigen binding proteins are provided.

In certain embodiments, acceptable formulation materials preferably are nontoxic to recipients at the dosages and concentrations employed. In certain embodiments, the pharmaceutical composition contain formulation materials for modifying, maintaining or preserving, for example, the pH, osmolarity, viscosity, clarity, color, isotonicity, odor, sterility, stability, rate of dissolution or release, adsorption or penetration of the composition. In such embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. See, REMINGTON'S PHARMACEUTICAL SCIENCES, 18″ Edition, (A. R. Genrmo, ed.), 1990, Mack Publishing Company.

In certain embodiments, the optimal pharmaceutical composition will be determined by one skilled in the art depending upon, for example, the intended route of administration, delivery format and desired dosage. See, for example, REMINGTON'S PHARMACEUTICAL SCIENCES, supra. In certain embodiments, such compositions influence the physical state, stability, rate of in vivo release and rate of in vivo clearance of the antigen binding proteins of the invention. In certain embodiments, the primary vehicle or carrier in a pharmaceutical composition is either aqueous or non-aqueous in nature. For example, a suitable vehicle or carrier is water for injection, physiological saline solution or artificial cerebrospinal fluid, possibly supplemented with other materials common in compositions for parenteral administration. Neutral buffered saline or saline mixed with serum albumin are further exemplary vehicles. In specific embodiments, pharmaceutical compositions comprise Tris buffer of about pH 7.0-8.5, or acetate buffer of about pH 4.0-5.5, and further includes sorbitol or a suitable substitute therefor. In certain embodiments of the invention, IL-17RA antigen binding protein compositions are prepared for storage by mixing the selected composition having the desired degree of purity with optional formulation agents (REMINGTON'S PHARMACEUTICAL SCIENCES, supra) in the form of a lyophilized cake or an aqueous solution. Further, in certain embodiments, the IL-17RA antigen binding protein product is formulated as a lyophilizate using appropriate excipients such as sucrose.

The pharmaceutical compositions of the invention can be selected for parenteral delivery. Alternatively, the compositions are selected for topical administration, for inhalation or for delivery through the digestive tract, such as orally. Preparation of such pharmaceutically acceptable compositions is within the skill of the art.

The formulation components are present preferably in concentrations that are acceptable to the site of administration. In certain embodiments, buffers are used to maintain the composition at physiological pH or at a slightly lower pH, typically within a pH range of from about 5 to about 8.

When parenteral administration is contemplated, the therapeutic compositions for use in this invention may be provided in the form of a pyrogen-free, parenterally acceptable aqueous solution comprising the desired IL-17RA antigen binding protein in a pharmaceutically acceptable vehicle. A particularly suitable vehicle for parenteral injection is sterile distilled water in which the IL-17RA antigen binding protein is formulated as a sterile, isotonic solution, properly preserved. In certain embodiments, the preparation involves the formulation of the desired molecule with an agent, such as injectable microspheres, bio-erodible particles, polymeric compounds (such as polylactic acid or polyglycolic acid), beads or liposomes, that provide controlled or sustained release of the product which is be delivered via depot injection.

Pharmaceutical compositions used for in vivo administration are typically provided as sterile preparations. Sterilization can be accomplished by filtration through sterile filtration membranes. When the composition is lyophilized, sterilization using this method is conducted either prior to or following lyophilization and reconstitution. Compositions for parenteral administration can be stored in lyophilized form or in a solution. Parenteral compositions generally are placed into a container having a sterile access port, for example, an intravenous solution bag or vial having a stopper pierceable by a hypodermic injection needle.

Aspects of the invention includes self-buffering IL-17RA antigen binding protein formulations, which can be used as pharmaceutical compositions, as described in international patent application WO 06138181A2 (PCT/US2006/022599), which is incorporated by reference in its entirety herein. One embodiment provides self-buffering IL-17RA antigen binding protein formulations comprising an IL-17RA antigen binding protein in which the total salt concentration is less than 150 mM.

One embodiment provides IL-17RA antigen binding protein formulations comprising an IL-17RA antigen binding protein wherein the concentration of the IL-17RA antigen binding protein is between approximately: 20 and 400, or 20 and 300, or 20 and 250, or 20 and 200, or 20 and 150 mg/ml, optionally between approximately 20 and 400 mg/ml, optionally between approximately 20 and 250, and optionally between approximately 20 and 150 mg/ml.

One embodiment provides IL-17RA antigen binding protein formulations comprising an IL-17RA antigen binding protein wherein the pH maintained by the buffering action of the IL-17RA antigen binding protein is between approximately: 3.5 and 8.0, or 4.0 and 6.0, or 4.0 and 5.5, or 4.0 and 5.0, optionally between approximately 3.5 and 8.0, and optionally between approximately 4.0 and 5.5.

One embodiment provides IL-17RA antigen binding protein formulations comprising an IL-17RA antigen binding protein wherein the salt concentration is less than: 150 mM or 125 mM or 100 mM or 75 mM or 50 mM or 25 mM, optionally 150 mM, optionally 125 mM, optionally 100 mM, optionally 75 mM, optionally 50 mM, and optionally 25 mM.

One embodiment provides IL-17RA antigen binding protein formulations comprising an IL-17RA antigen binding protein and one or more pharmaceutically acceptable salts; polyols; surfactants; osmotic balancing agents; tonicity agents; anti-oxidants; antibiotics; antimycotics; bulking agents; lyoprotectants; anti-foaming agents; chelating agents; preservatives; colorants; analgesics; or additional pharmaceutical agents.

One embodiment provides IL-17RA antigen binding protein formulations comprising an IL-17RA antigen binding protein and one or more pharmaceutically acceptable polyols in an amount that is hypotonic, isotonic, or hypertonic, preferably approximately isotonic, particularly preferably isotonic, such as but not limited to any one or more of sorbitol, mannitol, sucrose, trehalose, or glycerol, optionally approximately 5% sorbitol, 5% mannitol, 9% sucrose, 9% trehalose, or 2.5% glycerol.

One embodiment provides IL-17RA antigen binding protein formulations comprising an IL-17RA antigen binding protein further comprising a surfactant, preferably one or more of polysorbate 20, polysorbate 80, other fatty acid esters of sorbitan, polyethoxylates, and poloxamer 188, preferably polysorbate 20 or polysorbate 80, optionally approximately 0.001 to 0.1% polysorbate 20 or polysorbate 80, optionally approximately 0.002 to 0.02% polysorbate 20 or polysorbate 80, or optionally 0.002 to 0.02% polysorbate 20 or polysorbate 80.

One embodiment provides IL-17RA antigen binding protein formulations comprising an IL-17RA antigen binding protein wherein the formulation is sterile and suitable for treatment of a human or non-human subject.

As discussed above, certain embodiments provide IL-17RA antigen binding protein compositions, particularly pharmaceutical IL-17RA antigen binding protein compositions, that comprise, in addition to the IL-17RA antigen binding protein, one or more excipients such as those illustratively described in this section and elsewhere herein. Excipients can be used in the invention in this regard for a wide variety of purposes, such as adjusting physical, chemical, or biological properties of formulations, such as adjustment of viscosity, and or processes of the invention to improve effectiveness and or to stabilize such formulations and processes against degradation and spoilage due to, for instance, stresses that occur during manufacturing, shipping, storage, pre-use preparation, administration, and thereafter.

Embodiments of the IL-17RA antigen binding protein formulations further comprise one or more preservatives. Preservatives are necessary when developing multi-dose parenteral formulations that involve more than one extraction from the same container. Their primary function is to inhibit microbial growth and ensure product sterility throughout the shelf-life or term of use of the drug product. Commonly used preservatives include benzyl alcohol, phenol and m-cresol. Although preservatives have a long history of use with small-molecule parenterals, the development of protein formulations that includes preservatives can be challenging. Preservatives almost always have a destabilizing effect (aggregation) on proteins, and this has become a major factor in limiting their use in multi-dose protein formulations. To date, most protein drugs have been formulated for single-use only. However, when multi-dose formulations are possible, they have the added advantage of enabling patient convenience, and increased marketability. A good example is that of human growth hormone (hGH) where the development of preserved formulations has led to commercialization of more convenient, multi-use injection pen presentations. At least four such pen devices containing preserved formulations of hGH are currently available on the market. Norditropin® (liquid, Novo Nordisk), Nutropin AQ® (liquid, Genentech) & Genotropin (lyophilized—dual chamber cartridge, Pharmacia & Upjohn) contain phenol while Somatrope® (Eli Lilly) is formulated with m-cresol.

IL-17RA antigen binding protein formulations generally will be designed for specific routes and methods of administration, for specific administration dosages and frequencies of administration, for specific treatments of specific diseases, with ranges of bio-availability and persistence, among other things.

Formulations thus are designed in accordance with the invention for delivery by any suitable route, including but not limited to orally, aurally, opthalmically, rectally, and vaginally, and by parenteral routes, including intravenous and intraarterial injection, intramuscular injection, and subcutaneous injection. For example, a dose of the composition of the invention is delivered by subcutaneous injection by autoinjector syringe administered at time “0” (the first administration), at one week post time “0”, and then administered every two weeks following the week one administration. In particular, an antibody or any other IL-17RA antigen binding protein can be used to treat nail or scalp psoriasis in adult and/or juvenile patients at a dose of 70 mg per dose delivered by subcutaneous injection by autoinjector syringe administered at time “0” (the first administration), at one week post time “0”, and then administered every two weeks following the week one administration. An antibody or any other IL-17RA antigen binding protein can be used to treat nail or scalp psoriasis in adult and/or juvenile patients at a dose of 140 mg per dose delivered by subcutaneous injection by autoinjector syringe administered at time “0” (the first administration), at one week post time “0”, and then administered every two weeks following the week one administration. An antibody or any other IL-17RA antigen binding protein can be used to treat nail or scalp psoriasis in adult and/or juvenile patients at a dose of 210 mg per dose delivered by subcutaneous injection by autoinjector syringe administered at time “0” (the first administration), at one week post time “0”, and then administered every two weeks following the week one administration. An antibody or any other IL-17RA antigen binding protein can be used to treat nail or scalp psoriasis in adult and/or juvenile patients, and in particular plaque psoriasis, generalized pustular psoriasis and psoriatic erythroderma at a dose of 280 mg per dose delivered by subcutaneous injection by autoinjector syringe administered at time “0” (the first administration), at one week post time “0”, and then administered every two weeks following the week one administration.

The therapeutically effective amount of an IL-17RA antigen binding protein-containing pharmaceutical composition to be employed will depend, for example, upon the therapeutic context and objectives. One skilled in the art will appreciate that the appropriate dosage levels for treatment will vary depending, in part, upon the molecule delivered, the indication for which the IL-17RA antigen binding protein is being used, the route of administration, and the size (body weight, body surface or organ size) and/or condition (the age and general health) of the patient. In certain embodiments, the clinician titers the dosage and modify the route of administration to obtain the optimal therapeutic effect. A typical dosage rangse from about 0.1 μg/kg to up to about 30 mg/kg or more, depending on the factors mentioned above. In specific embodiments, the dosage may range from 0.1 μg/kg up to about 30 mg/kg, optionally from 1 μg/kg up to about 30 mg/kg or from 10 μg/kg up to about 5 mg/kg.

Dosing frequency will depend upon the pharmacokinetic parameters of the particular IL-17RA antigen binding protein in the formulation used. Typically, a clinician administers the composition until a dosage is reached that achieves the desired effect. The composition is therefore administered as a single dose, or as two or more doses (which may or may not contain the same amount of the desired molecule) over time, or as a continuous infusion via an implantation device or catheter. In the treatment method of the present invention, an administration of the antibody or the antibody fragment thereof is not particularly limited, however, the administration is desirably performed at day 1, weeks 1 and 2 and may further be continued after week 2 every other week. Alternatively, continuous administrations may be performed at the starting date of the administration every other week. The dosing period of the antibody or the antibody fragment thereof is not particularly limited, however, the period is desirably 10 weeks or more, more desirably 50 weeks or more, further more desirably 62 weeks or more from the starting date of the administration. In addition, the dosing period may include a rest period. In the treatment method of the present invention, the antibody or the antibody fragment thereof may be administered one time. Further refinement of the appropriate dosage is routinely made by those of ordinary skill in the art and is within the ambit of tasks routinely performed by them. Appropriate dosages are ascertained through use of appropriate dose-response data. In the treatment method of the present invention or the therapeutic agent of the present invention, a dose of the antibody or the antibody fragment thereof per one time is not particularly limited, however, is desirably 70 mg or more, 140 mg or more, 210 mg or more or 280 mg or more. Further, the dose may be increased or reduced during the continuous administration of the antibody or the antibody fragment thereof. In certain embodiments, the antigen binding proteins of the invention can be administered to patients throughout an extended time period. Chronic administration of an antigen binding protein of the invention minimizes the adverse immune or allergic response commonly associated with antigen binding proteins that are not fully human, for example an antibody raised against a human antigen in a non-human animal, for example, a non-fully human antibody or non-human antibody produced in a non-human species.

The route of administration of the pharmaceutical composition is in accord with known methods, e.g., orally, through injection by intravenous, intraperitoneal, intracerebral (intra-parenchymal), intracerebroventricular, intramuscular, intra-ocular, intraarterial, intraportal, or intralesional routes; by sustained release systems or by implantation devices. In certain embodiments, the compositions are administered by bolus injection or continuously by infusion, or by implantation device.

The composition also may be administered locally via implantation of a membrane, sponge or another appropriate material onto which the desired molecule has been absorbed or encapsulated. In certain embodiments, where an implantation device is used, the device is implanted into any suitable tissue or organ, and delivery of the desired molecule via diffusion, timed-release bolus, or continuous administration.

It also may be desirable to use IL-17RA antigen binding protein pharmaceutical compositions according to the invention ex vivo. In such instances, cells, tissues or organs that have been removed from the patient are exposed to IL-17RA antigen binding protein pharmaceutical compositions after which the cells, tissues and/or organs are subsequently implanted back into the patient.

In particular, IL-17RA antigen binding proteins can be delivered by implanting certain cells that have been genetically engineered, using methods such as those described herein, to express and secrete the polypeptide. In certain embodiments, such cells, animal or human cells, and may be autologous, heterologous, or xenogeneic. In certain embodiments, the cells may be immortalized. In other embodiments, in order to decrease the chance of an immunological response, the cells may be encapsulated to avoid infiltration of surrounding tissues. In further embodiments, the encapsulation materials are typically biocompatible, semi-permeable polymeric enclosures or membranes that allow the release of the protein product(s) but prevent the destruction of the cells by the patient's immune system or by other detrimental factors from the surrounding tissues.

All references cited within the body of the instant specification are hereby expressly incorporated by reference in their entirety.

### EXAMPLES

The following example, including the experiments conducted and the results achieved, are provided for illustrative purposes only and are not to be construed as limiting the invention.

### Example 1

### Brodalumab Effectively Treats Nail and Scalp Psoriasis

### Study Parameters and Overall Schema

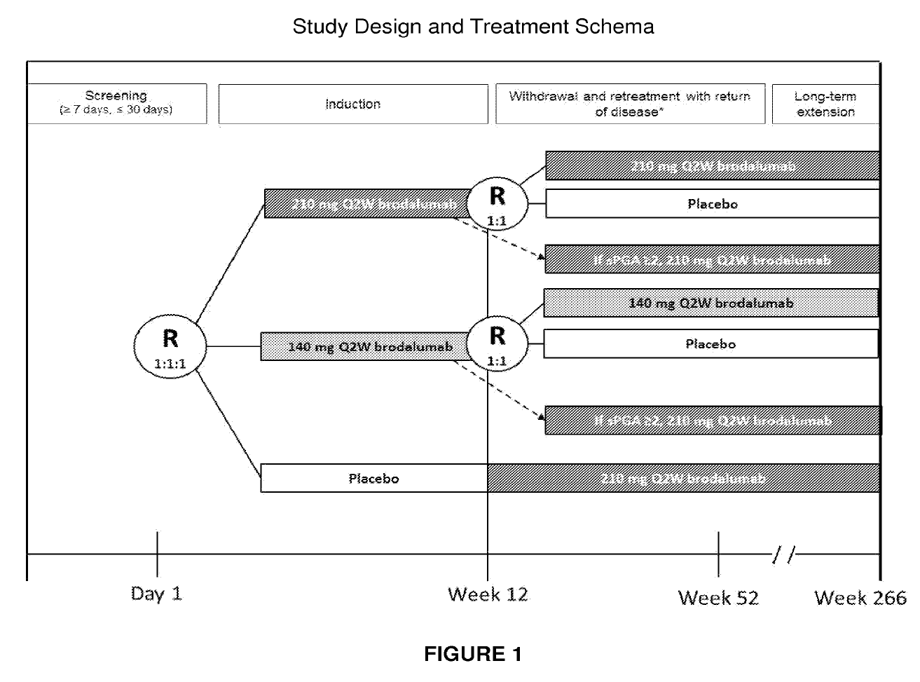

Scalp and nail psoriasis were evaluated as part of the brodalumab phase 3 clinical trial which enrolled subjects with moderate to severe psoriasis. After a screening period, the study began with a 12-week double-blind, placebo-controlled induction phase. In this phase, the subjects were randomized in a 1:1:1 ratio to either received brodalumab at 210 mg Q2W, brodalumab at 140 mg Q2W or the placebo. Randomization was stratified by baseline total body weight (≦100kg; >100 kg), by prior biologic use and by geographic region. Subjects with prior biologic use were capped at 50% of the study population. After 12 weeks, the treatment was withdrawn and retreatment was initiated with either the same dose or a placebo with the return of disease through 52 weeks. The study continued for 266 weeks. A summary of the overall study schema is provided in FIG. 1.

Study subjects had stable moderate to severe plaque psoriasis for at least 6 months. Subjects were candidates to receive a biologic therapy for psoriasis, in the opinion of the investigator, according to regional labeling and had psoriasis that involves body surface area ≧10%, PASI≧12, and sPGA≧3 at screening and baseline visits. In particular, the subjects with a Psoriasis Scalp Severity Index (PSSI)≧15 (out of a potential 72) and affected Scalp Surface Area (SSA)≧30% at baseline were followed at scheduled visits. In subjects with nails involved with psoriasis, each nail was scored at baseline to determine the worst nail (i.e., the nail with the highest Nail Psoriasis Severity Index (NAPSI) score). In subjects whose worst nail has a minimum NAPSI score of 6 at baseline, that nail (the target nail) was followed for the remainder of the study.

### Scalp Assessments

To determine the PSSI and SSA scores, assessments were completed by the same assessor performing the PASI assessment. The PSSI is a scalp-specific modification of the PASI, based on the extent of involvement and the severity of erthyema, induration and desquamation. The SSA numerical score (0% to 100%) measures the assessor's assessment of the proportion of the subject's total SSA involved with psoriasis. Those subjects with a PSSI≧15 (out of a potential 72) and an SSA≧30% at baseline were followed for these assessments at scheduled visits.

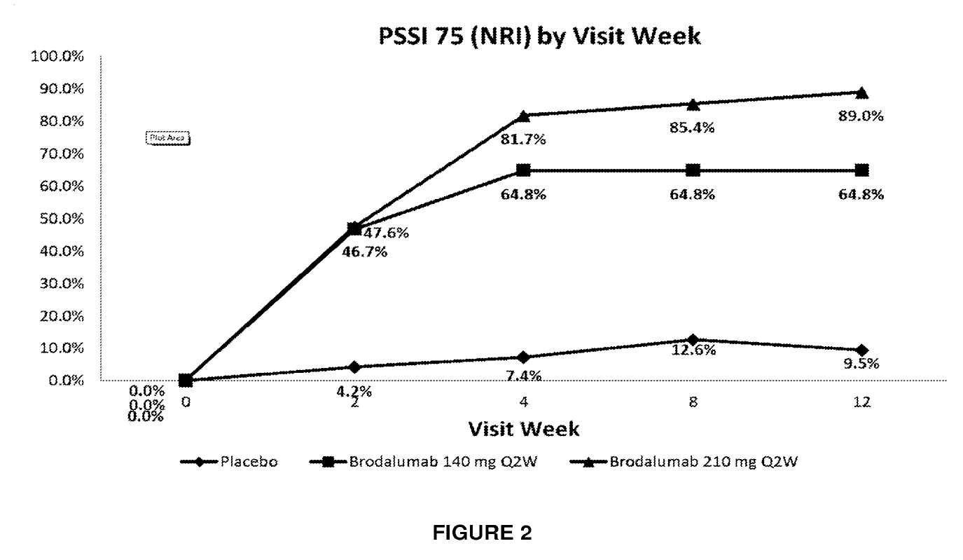

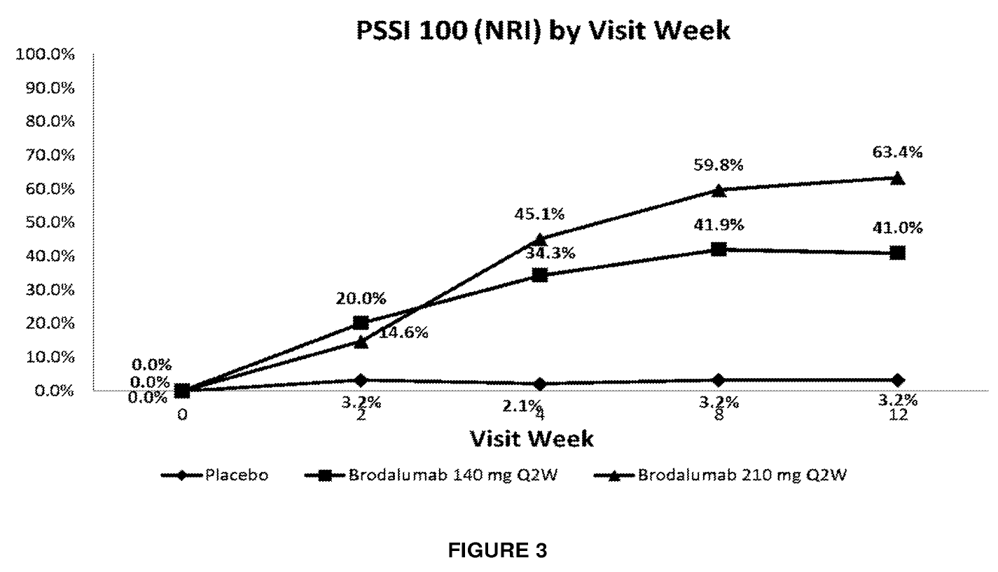

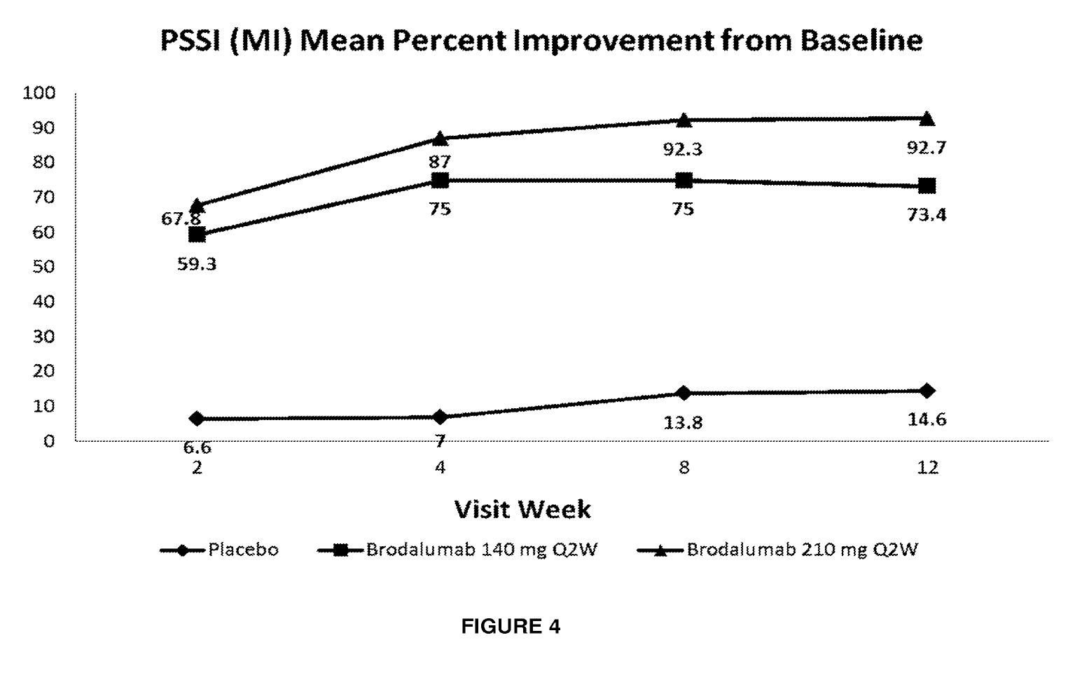

The scalp psoriasis PSSI- 75 (NRI) and the PSSI-100 were measured every two weeks. The PSSI-75 orPSSI-100 refers to that percent of patients who achieved 75% or 100% of the PSSI percent improvement from baseline. As shown in FIGS. 2 and 3, brodalumab at doses140 mg and 210 mg Q2W is more efficacious than placebo at treating scalp psoriasis as measured by the PSSI and SSA at week 12. The PSSI (MI) percent improvement from baseline is provided in FIG. 4.

### Nail Assessments

The NAPSI scale is objective, numeric and reproducible grading system for nail psoriasis that incorporates the many different features of nail psoriasis. For assessments in the study (including selection of target nail), a nail was graded using the NAPSI scale by first dividing the nail with imaginary horizontal and vertical lines into 4 quarters. The following eight clinical features of nail psoriasis are then scored based on the number of quarters in which the feature is present (0 to 4) to arrive at a NAPSI score of 0 to 32 for each nail: pitting, leukonychia, red spots in lunula, nail plate crumbling, oil drop (salmon patch) discoloration, onycholysis, nail bed hyperkeratosis, splinter hemorrhages.

In randomized subjects with nails involving psoriasis, each nail was scored at baseline to determine the worst nail (i.e., the nail with the highest NAPSI score). Those subjects whose nail has a minimum NAPSI score of 6 at baseline will have this nail (the target nail) followed for the remainder of the study. If multiple nails have the same worst score, only 1 target nail was followed.

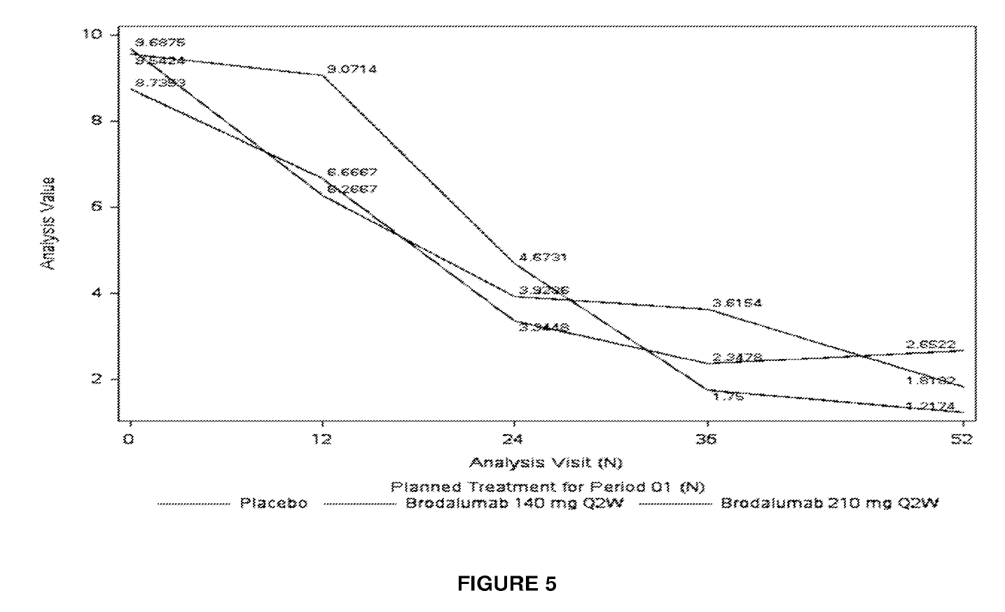

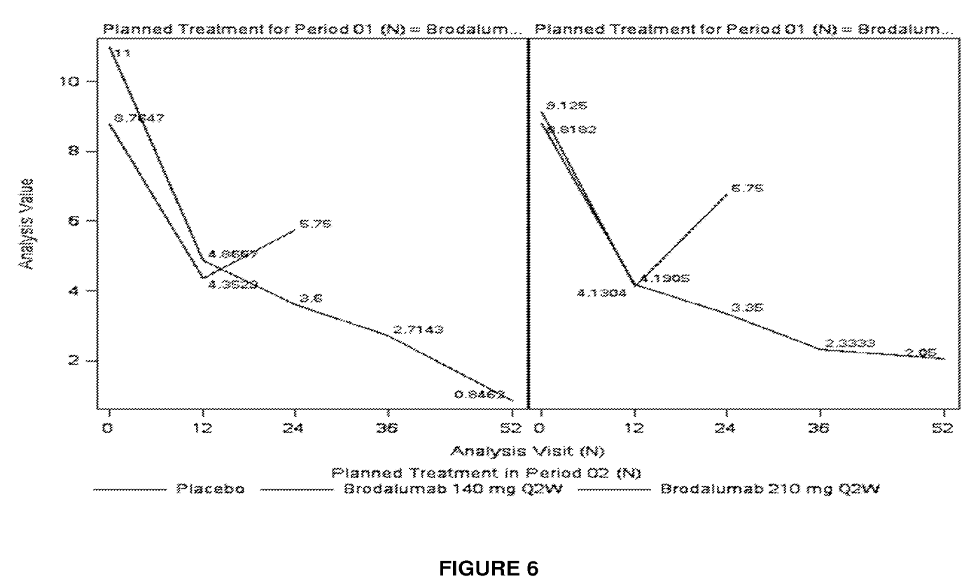

The NAPSI score (as observed) by treatment group in the induction phase is provided in FIG. 5. The NAPSI score (as observed) for non-rerandomized subjects by treatment group in the induction phase through week 52 is provided in FIG. 6. Brodalumab at dose 140 mg and 210 mg Q2W was more efficacious than placebo at treating nail psoriasis as measured by the NAPSI at week 12. Brodalumab at doses 140 mg and 210 mg Q2W was effective at treating nail psoriasis as measured by the NAPSI over 52 weeks.

### Adverse Events

The subject incidence rates for the placebo-controlled 12 week induction phase in the overall study population are set out in Table 1 below. The most frequently reported adverse event (occurring in ≧5% in any treatment group) were nasopharyngitis, upper respiratory tract infection, and headache. There were no meaningful imbalances in the AE rates in the induction phase between placebo and brodalumab treatment arms.

TABLE 1

| | Brodalumab | | | |
|---|---|---|---|---|
| | | | 210 mg | |
| | Placebo | 140 mg Q2W | Q2W | All |
| | (N = 220) | (N = 219) | (N = 222) | (N = 441) |
| All treatment- | 112 (50.9) | 126 (57.5) | 131 (59.0) | 257 (58.3) |
| emergent | | | | |
| adverse | | | | |
| events - n (%) | | | | |
| Grade ≧2 | 62 (28.2) | 70 (32.0) | 75 (33.8) | 145 (32.9) |
| Grade ≧3 | 9 (4.1) | 8 (3.7) | 15 (6.8) | 23 (5.2) |
| Serious | 3 (1.4) | 6 (2.7) | 4 (1.8) | 10 (2.3) |
| Fatal adverse | 0 (0.0) | 0 (0.0) | 0 (0.0) | 0 (0.0) |
| events | | | | |

The exposure adjusted event rates through week 52 in the overall study population are set out in Table 2 below. The term “Subj-yr” refers to the total subject years of exposure through week 52 excluding periods of placebo exposure in the induction or withdrawal phases. The term “n” refers to the number of adverse events reported. The term “r” refers to exposure-adjusted event rate per 100 subject-years (n/subj-yr*100). Adverse events reported during periods of placebo exposure were excluded. The adverse events and exposure in subjects randomized to placebo in the induction phase were summarized after their first dose of 210 mg Q2W at or after week 12 under the constant 210 mg Q2W dose group. Multiple occurrences of the same event for a subject were counted as multiple events.

Treatment groups are defined as planned through week 52. Constant Dose groups are subjects who received the same dose across all phases after first brodalumab administration. The Combination of 140/210 groups are those subjects with planned treatment with both 140 mg Q2W and 210 mg Q2W through week 52. The Mixed Dosing group refers to subjects that were re-randomized to placebo in withdrawal period.

TABLE 2

| | Brodalumab | | | | |
|---|---|---|---|---|---|
| | | Combination | Constant | Constant | |
| | Mixed | 140/210 mg | 140 mg | 210 mg | |
| | dosing | Q2W | Q2W | Q2W | All |
| | (Subj-yr = | (Subj-yr = | (Subj-yr = | (Subj-yr = | (Subj-yr = |
| | 105.2) | 89.1) | 51.2) | 271.8) | 517.3) |
| | (N = 143) | (N = 99) | (N = 61) | (N = 345) | (N = 648) |
| | n (r) | n (r) | n (r) | n (r) | n (r) |
| All treatment— | 343 (326.1) | 347 (389.2) | 184 (359.2) | 1034 (380.4) | 1908 (368.8) |
| emergent | | | | | |
| adverse | | | | | |
| events—n (%) | | | | | |
| Grade ≧ 2 | 140 (133.1) | 167 (187.3) | 79 (154.2) | 460 (169.2) | 846 (163.5) |
| Grade ≧ 3 | 12 (11.4) | 13 (14.6) | 11 (21.5) | 55 (20.2) | 91 (17.6) |
| Serious | 6 (5.7) | 11 (12.3) | 5 (9.8) | 27 (9.9) | 49 (9.5) |
| Leading to | 1 (1.0) | 1 (1.1) | 4 (7.8) | 10 (3.7) | 16 (3.1) |
| discontinuation | | | | | |
| of IP | | | | | |
| Leading to | 1 (1.0) | 1 (1.1) | 3 (5.9) | 9 (3.3) | 14 (2.7) |
| discontinuation | | | | | |
| from study | | | | | |
| Fatal adverse | 1 (1.0) | 0 (0.0) | 0 (0.0) | 3 (1.1) | 4 (0.8) |
| events | | | | | |

### Example 2

### Phase II Clinical Trial of Brodalumab in Japan

A randomized, double blind, placebo-controlled phase II clinical trial was carried out in Japan to investigate the effectiveness of the human monoclonal AM-14 (known an brodalumab) in subjects with moderate to severe plaque psoriasis. 151 subjects suffering from moderate to severe psoriasis, which satisfied the conditions provided in Table 3, were randomly divided into four groups. The subjects in each group were administered one of the following dose of brodalumab: 0 mg (placebo), 70 mg, 140 mg or 210 mg. Brodalumab was administered as a subcutaneous injection on day 1 and at week 1, week 2, week 4, week 6, week 8 and week 10.

TABLE 3

| | |
|---|---|
| Ages Eligible for Study: | 20 Years to 70 Years |
| Genders Eligible for Study: | Both |
| Accepts Healthy Volunteers: | No |

Criteria

1) Inclusion Criteria:
Subject had stable moderate to severe plaque psoriasis for at least
6 months.
Subject received at least one previous phototherapy or systemic psoriasis
therapy or has been a candidate to receive phototherapy or systemic
psoriasis therapy in the opinion of the investigator.
Subject has involved BSA ≧10% and PASI ≧12 at screening and
at baseline.
2) Exclusion Criteria:
Subject diagnosed with erythrodermic psoriasis, pustular psoriasis,
medication-induced, or medication-exacerbated psoriasis.
Evidence of skin conditions at the time of the screening visit (eg, eczema)
that would interfere with evaluations of the effect of investigational
product on psoriasis.
Subject had any active Common Terminology Criteria for Adverse Events
(CTCAE) grade 2 or higher infection
Subject a significant concurrent medical condition or laboratory
abnormalities, as defined in the study protocol.
Subject used the following therapies within 14 days of the first
dose: topical calcineurin inhibitors including tacrolimus, topical vitamin
A, activated form D3 or activated form D3 analogue preparations,
weak through strong topical steroids (excluding application on the
scalp, axillae, and groin)
Subject used the following therapies within 28 days of the first dose: any
other systemic psoriasis therapy (eg, vitamin A, calcineurin inhibitors,
methotrexates, steroids), UVA therapy (with or without psoralen), very
strong or strongest topical steroid, tar therapy
Subject used the following therapies within 3 months of the first dose:
adalimumab, etanercept, infliximab, or live vaccines
Subject used ustekinumab within 6 months of the first dose
Subject previously used an anti-interleukin-17 biologic therapy

The degree of severity of psoriasis symptoms on the nail and the scalp of the subjects in each administered group was measured on the first day brodalumab was administered to the subject (hereinafter, described as week 0 from the first administration day 1 from the first administration), Week 0 or day 1) and at week 12 after the first administration. The NAPSI and PSSI score were respectively calculated based on the descriptions by Rich et al. (J Am Acad Dermatol; vol. 49, number 2, p. 206-212) and Leonardi et al. (J Engl J Med 2012; 366: 1190-9).

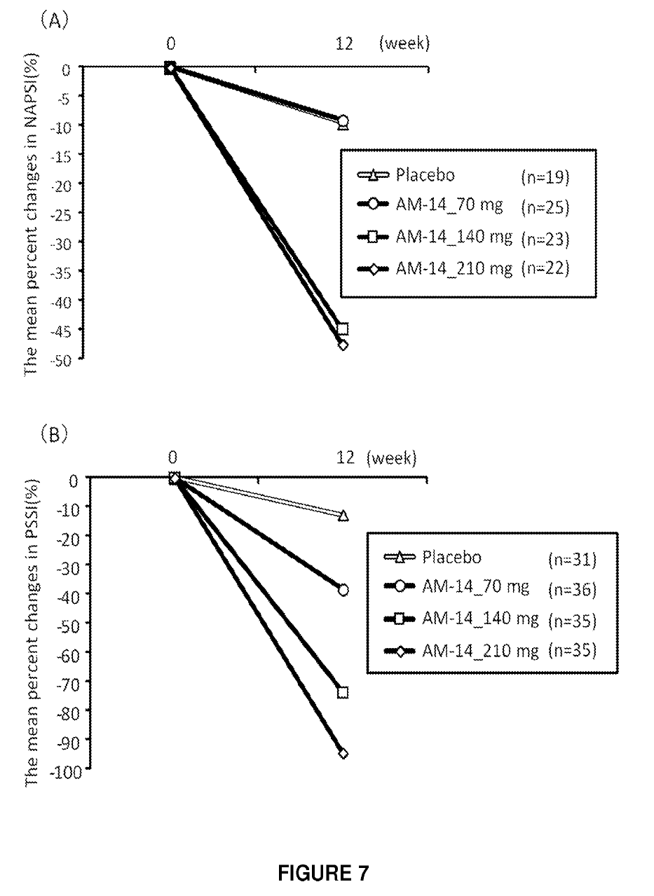

FIG. 7 provides the mean percent change in the NAPSI (panel A) and PSSI (panel B) from baseline and demonstrates that brodalumab had a therapeutic effect on nail (FIG. 7A) and scalp psoriasis (FIG. 7B). The mean percent change from baseline is calculated according to formula 1 and the baseline set as day 1 of this trial in this analysis. The obtained mean percent change in the NAPSI score in the placebo administered group was a reduction by 9.6%, while the mean percent change in the NAPSI scores from baseline was a reduction by 9.1%, 44.9% and 47.3% in response to 70 mg 140 mg and 210 mg of brodalumab, respectively. Thus, the NAPSI scores were greatly reduced in the subjects receiving 140 mg and 210 mg of brodalumab as compared to the placebo group.

In addition, a mean percent change in the PSSI score from baseline in the placebo administered group was reduced by 12.6%, while the men percent change in the PSSI from baseline was a reduction by 38.3%, 73.8% and 94.5% in response to 70 mg 140 mg and 210 mg of brodalumab, respectively. Thus, the PSSI scores were greatly reduced in the subjects receiving 70 mg, 140 and 210 mg of brodalumab as compared to the placebo group. This trial demonstrated that brodalumab improved the degree of severity of nail and scalp psoriasis in subjects suffering from moderate to severe plaque psoriasis.

### Example 3

### Phase III Clinical Trial of Brodalumab in Japan

A randomized, placebo-controlled, randomized phase III clinical trial was carried out in Japan to investigate the safety and effectiveness of the human monoclonal antibody AM-14 (known an brodalumab). The trial was double-blind and randomized for 4 week after the start of administration of brodalumab, followed by an open-labeled, randomized extension study from week 4 to week 52. The subjects suffering from moderate to severe plaque psoriasis that completed the trial described in Example 2 and satisfied the conditions presented in Table 4 were selected as subjects for this phase III trial. This phase III study was started on the date of the completion of the phase II trial (Example 2).

145 subjects were enrolled in the trial (133 subjects completed the trial) and were divided into 2 groups. The subjects receiving 140 mg or 210 mg in the phase II trial were administered the same amount of brodalumab by subcutaneous injection every other week in the phase III trial. The subjects administered with 0 mg (placebo) or 70 mg of AM-14 in the phase II study were randomly divided in two groups in the phase III trial, and each group was administered with 140 mg or 210 mg of AM-14 in weeks 0, 1, and 2. After week 2, the antibody was administered every other week. AM-14 administration was continued for 50 weeks, and Phase II trial was completed on week 52.

TABLE 4

| | |
|---|---|
| Ages Eligible for Study: | 20 Years to 70 Years |
| Genders Eligible for Study: | Both |
| Accepts Healthy Volunteers: | No |

Criteria

Inclusion Criteria:
Subject has voluntarily signed the written informed consent form to
participate in this study
Subject has completed the week 12 evaluation of Phase II trial
(Example 2)
Exclusion Criteria:
Subject has had a serious infection, defined as requiring systemic
treatment with antibiotics or antivirals (excluding oral administration)
Subject had been judged to be ineligible for participation in the study by
the investigators/subinvestigators

The degree of severity of psoriasis symptoms on the nail and the scalp of each subject was measured on day 1 of phase II trial (Example 2) and at week 52 of the phase III trial. NAPSI and PSSI scores were calculated in the same manner as described in Example 2.

Table 5 and Table 6 showed the mean percent change in NAPSI and PSSI score from baseline, respectively. The mean percent change from baseline was calculated according to formula 1. In this analysis, day 1 of the Phase II trial (Example 2) was set as baseline.

TABLE 5
The mean percent change in NAPSI score from baseline

| | Phase II | Phase III | | | | |
|---|---|---|---|---|---|---|
| | Day 1 | Week | Week | Week | Week | Week |
| | (baseline) | 0 | 12 | 24 | 36 | 52 |
| 140 mg of | 0% | −29.7% | −51.7% | −74.8% | −73.1% | −73.7% |
| brodalumab | | | | | | |
| (N = 43) | | | | | | |
| 210 mg of | 0% | −26.4% | −73.0% | −85.4% | −87.7% | −88.1% |
| brodalumab | | | | | | |
| (N = 46) | | | | | | |

TABLE 6
The mean percent change in PSSI score from baseline

| | Phase II | Phase III | | | | |
|---|---|---|---|---|---|---|
| | Day 1 | Week | Week | Week | Week | Week |
| | (baseline) | 0 | 12 | 24 | 36 | 52 |
| 140 mg of | 0% | −51.4% | −86.4% | −83.8% | −82.4% | −84.6% |
| brodalumab | | | | | | |
| (N = 68) | | | | | | |
| 210 mg of | 0% | −60.4% | −90.4% | −87.4% | −82.8% | −82.6% |
| brodalumab | | | | | | |
| (N = 69) | | | | | | |

Thus, the NAPSI and PSSI score greatly decreased from day 1 of the phase II trial to the date of completion of the phase III trial in subjects receiving 140 mg and 210 mg of brodalumab. The degree of severity of the nail and scalp psoriasis greatly improved in plaque psoriasis patients administered with brodalumab.

### Example 4

### Phase III Clinical Trial of Brodalumab in Japan

Another Phase III Clinical Trial of brodalumab was conducted in Japan in subjects with generalized pustular psoriasis (GPP) or psoriatic erythroderma (PsE). A summary of this clinical trials was described in Table 7.

TABLE 7

| | | No. |
|---|---|---|
| | | Subjects |
| Indication, Design | Dose, Duration | Enrolled |
| Indication; | Dose; 140 mg (dosage | 12 |
| generalized pustular | may be increased to 210 mg | patients |
| psoriasis (GPP) or | in case of insufficient | (GPP), |
| psoriatic | efficacy) | 18 |
| erythroderma (PsE) | Duration; 50 weeks | patients |
| Design; Open-label, | | (PsE) |
| Uncontrolled Study | | |

Pustular psoriasis patients or psoriatic erythroderma patients that satisfy the conditions presented in Table 8 were selected as clinical trial subjects, and evaluated for the safety and efficacy or the like of brodalumab after chronic administration. Each subject was administered with 140 mg of brodalumab on day 1, in week 1 and in week 2, and every other week from week 2 to week 50. For subjects in which a sufficient effect was not obtained by the end of week 4, the dose was increased and 210 mg of AM-14 was administered every other week from week 6.

TABLE 8

| | |
|---|---|
| Ages Eligible for Study: | 18 Years and older |
| Genders Eligible for Study: | Both |
| Accepts Healthy Volunteers: | No |

Criteria

1) Inclusion Criteria:
Subject has signed voluntarily the written informed consent form to
participate in this study.
Subject has been diagnosed as pustular psoriasis or psoriatic erythroderma.
Subject has received at least one previous phototherapy or systemic
psoriasis therapy or has been a candidate to receive phototherapy or
systemic psoriasis therapy in the opinion of the investigator.
2) Exclusion Criteria:
Subject with psoriatic erythroderma has involved Body surface area (BSA)
of lesion <80% at baseline.
Subject diagnosed with guttate psoriasis, medication-induced or
medication-exacerbated psoriasis.
Evidence of skin conditions at the time of the screening visit (eg, eczema)
that would interfere with evaluations of the effect of AM-14 on psoriasis.
Subject has any active Common Terminology Criteria for Adverse Events
(CTCAE) grade 2 or higher infection
Subject has a significant concurrent medical condition or laboratory
abnormalities, as defined in the study protocol.
Subject has used Ultra Violet B (UVB) therapy within 14 days of the first
dose or Ultra Violet A (UVA) (with or without psoralen) within 28 days
of the first dose.
Subject has used etanercept, adalimumab, infliximab or ustekinumab
within 1 week, 2 weeks, 8 weeks or 12 weeks of the first dose,
respectively.
Subject has stopped ustekinumab or other anti-Interleukin (IL)-23
biologics therapy due to lack of efficacy
Subject has used live vaccine within 3 months of the first dose
Subject has previously used an anti-IL-17 biologic therapy

The degree of severity of psoriasis sympton on the nail and the scalp of each subject was measured on week 0, 12, 24, 36, 52 of this phase III trial. NAPSI and PSSI score were calculated in the same manner as described in Example 2.

Table 9 and Table 10 showed the mean percent change in NAPSI and PSSI score from baseline, respectively. The mean percent change was calculated according to formula 1. In this analysis Day 1(Week 0) of this trial was set as baseline.

TABLE 9
The mean percent change in NAPSI score from baseline

| | Week 0 | Week | | | |
|---|---|---|---|---|---|
| | (baseline) | 12 | Week 24 | Week 36 | Week 52 |
| Pustular | 0% | −47.6% | −59.2% | −60.7% | −67.5% |
| (N = 4) | | | | | |
| Erythroderma | 0% | −45.5% | −66.3% | −83.9% | −83.5% |
| (N = 13) | | | | | |

TABLE 10
The mean percent change in PSSI score from baseline

| | Week 0 | Week | | | |
|---|---|---|---|---|---|
| | (baseline) | 12 | Week 24 | Week 36 | Week 52 |
| Pustular | 0% | −77.7% | −87.5% | −95.2% | −96.1% |
| (N = 9) | | | | | |
| Erythroderma | 0% | −82.5% | −89.9% | −96.2% | −95.9% |
| (N = 18) | | | | | |

Thus, the NAPSI and PSSI score was greatly reduced in subjects receiving brodalumab. This trial demonstrated that brodalumab improved the degree of severity of nail and scalp psoriasis in subjects suffering from GPP or PsE.

What is claimed:

1. A method for treating nail or scalp psoriasis, comprising administering a composition comprising an antibody or fragment thereof to a patient wherein the antibody specifically binds to IL-17 Receptor A (IL-17RA) and has an antagonistic activity.

2. A method of according to claim 1, wherein the antibody or fragment thereof is selected from:
a. an antibody, comprising a light chain variable domain comprising the amino acid sequence of SEQ ID NO:7 and a heavy chain variable domain comprising the amino acid sequence of SEQ ID NO:8;
b. an antibody comprising the full length light chain amino acid sequence of SEQ ID NO: 9 and a full length heavy chain amino acid sequence of SEQ ID NO: 10 and
c. an antibody, comprising a light chain CDR1 comprising the amino acid sequence of SEQ ID NO:1, a light chain CDR2 comprising the amino acid sequence of SEQ ID NO:2, a light chain CDR3 comprising the amino acid sequence of SEQ ID NO:3, a heavy chain CDR1 comprising the amino acid sequence of SEQ ID NO:4, a heavy chain CDR2 comprising the amino acid sequence of SEQ ID NO:5, and a heavy chain CDR3 comprising the amino acid sequence of SEQ ID NO:6.

3. The method of claim 1 or 2, wherein the antibody or fragment thereof inhibits binding of IL-17A to said IL-17RA.

4. The method of any of the preceding claims wherein the patient is suffering from psoriasis.

5. The method of any of the preceding claims wherein the patient is suffering from plaque psoriasis, pustular psoriasis or psoriatic erythroderma.

6. The method of any of the preceding claims wherein the patient has a NAPSI score of at least 6 or a modified NAPSI score of at least 2 on one or more nails.

7. The method of any of the preceding claims wherein the patient has a PSSI score of at least 15.

9. The method of any of the preceding claims where the patient has a SSA score of at least 30%.

10. The method of treating nail psoriasis according to any of preceding claims, wherein the composition is administered at a dose of antibody or fragment thereof effective to reduce or maintain a NAPSI score of 6 or less or a mNAPSI score of 3 or less on an affected nail.

11. The method of treating scalp psoriasis according to any of preceding claims wherein the composition is administered at a dose of antibody or fragment thereof effective to reduce and maintain a PSSI score of 15 or less.

12. The method of any of the preceding claims wherein the composition administered comprises a dose of antibody that is 70 mg, 140mg, 210 mg or 280 mg.

13. A method of treating nail or scalp psoriasis or any of the preceding claims wherein the patient has less than 10% body surface area affected by psoriasis and additionally having nail or scalp psoriasis.

14. The method of any of the preceding claims wherein the patient has less than 10% body surface area affected by moderate to severe psoriasis, plaque psoriasis, pustular psoriasis or psoriatic erythroderma.

15. The method of claim 13 or 14 wherein the patient has about 7% or less body surface area affected by psoriasis.

16. The method of claim 13 or 14 wherein the patient has about 5% or less body surface area affected by psoriasis.

17. The method of claim 13 or 14 wherein the patient has about 3% or less body surface area affected by psoriasis.

18. The method of claim 13 or 14 wherein the patient has about 1% or less body surface area affected by psoriasis.

19. The method any one of the preceding claims, further comprising administering to said patient a second treatment.

20. The method of claim 19, wherein said second treatment is administered prior to, concurrent with, or subsequent to administration of said composition comprising said antibody.

21. The method of claim 19 or 20 wherein the second treatment is a topical treatment.

22. The method of claim 21 where in the topical treatment is selected from the group consisting fluorouracil, dithranol, tazarotene, cyclosporine, calcineneurin inhibitors, triamcinolone, fluocinonide, topical steroids, vitamin D₃, vitamin D₃ analogs, betamethasone dipropionate, betamethasone valerate, calcipotriol, clobetasol, XAMIOL, DAIVOBET, coal tar, urea, corticosteroids, retinoids, anthralin, topical methatrexate, keratolytics, salicylic acid, tofacitinab, apremilast, topical JAK inhibitors or a combination thereof.

23. The method of any one of claims 20-22, wherien the second treatment is selected from the group consisting of retinoids, acitretin cyclosporine, methotrexate, apremilast, tofacitinib, oral JAK inhibitors, oral PI3 kinase inhibitors, oral MAP kinase inhibitors, Fumaderm, fumarates, dimethyl fumarate, sulfasalazine, leflunomide, calcineurin inhibitors, azathioprine, thioguanine, hydroxyurea, hydroxychloroquine, sulfasalazine, antifungals or a combination thereof.

24. The method of any one of claims 20-23, wherein the second treatment an antibody or chimeric protein specific for TNF IL-17 IL-12/23 or IL-23.

25. The method of claim 24, wherein the antibody or chimeric protein is infliximab, adalimumab, etanercept, alefacept, ustekinumab, secukinumab, ixekizumab, guselkumab, antifungals or a combination thereof.

26. The method of any one of claims 20-25, wherein the second treatment is selected from the group consisting of is triamcinolone acetonide photochemotherapy, laser therapy, Excimer laser, oral/topical psorallen with UVA (PUVA), pulsed dye laser, radiation therapy, superficial radiotherapy, electron beam therapy, Grenz ray therapy, ermatome shaving, aloe vera extract, narrow band UV therapy UV therapy or a combination thereof.

27. The method of any one of the preceding claims, wherein said antibody is selected from the group consisting of:
a. a humanized antibody;
b. a chimeric antibody;
c. a monoclonal antibody;
d. an antigen-binding antibody fragment;
e. a single chain antibody;
f. a diabody;
g. a triabody;
h. a tetrabody;
i. a Fab fragment;
j. a F(ab')2 fragment;
k. an IgD antibody;
l. an IgE antibody;
m. an IgM antibody;
n. an IgG1 antibody;
o. an IgG2 antibody;
p. an IgG3 antibody; and
q.an IgG4 antibody.

28. The method of any one of the preceding claims, wherein said composition is a pharmaceutical composition.

29. The method of any one of the preceding claims, wherein said pharmaceutical composition further comprises a pharmaceutically acceptable diluent.

30. The method of claim 28 or 29 wherein the pharmaceutical composition comprises an aqueous solution of a glutamic acid buffer comprising a) said glutamic acid buffer comprises a glutamic acid concentration of 5-30 mM.+-.0.2 mM; b) said glutamic acid buffer comprises a pH of 4.5-5.2.+-.0.2; c) said formulation further comprises 2-4% proline (w/v) and 0.005-0.02% (w/v) polysorbate 20 and d) the antibody or fragment thereof at a concentration of 100 to 150 mg/ml.

31. The method of claim 30 wherein the pharmaceutical composition has an osmolarity of 275 to 325 osm.

32. The method of claim 30 or 31 wherein the pharmaceutical composition has a viscosity of 5 to 7 cP at 25° C.

33. The method of any one of the preceding claims, wherein said antibody is a human IgG2 monoclonal antibody.

34. Use of an antibody or fragment thereof for the preparation of a medicament for the treatment of nail or scalp psoriasis, wherein the antibody or fragment thereof specifically binds to IL-17receptor RA (IL-17RA) and has an antagonistic activity.

35. A composition for use in the treatment of nail or scalp psoriasis, wherein the composition comprises an antibody or fragment thereof that specifically binds to IL-17receptor RA (IL-17RA) and has an antagonistic activity.

36. The use or composition of claim 34 or 35, wherein the antibody or fragment thereof is selected from:
a. an antibody, comprising a light chain variable domain comprising the amino acid sequence of SEQ ID NO:7 and a heavy chain variable domain comprising the amino acid sequence of SEQ ID NO:8;
b. an antibody comprising the full length light chain amino acid sequence of SEQ ID NO: 9 and a full length heavy chain amino acid sequence of SEQ ID NO: 10 and
c. an antibody, comprising a light chain CDR1 comprising the amino acid sequence of SEQ ID NO:1, a light chain CDR2 comprising the amino acid sequence of SEQ ID NO:2, a light chain CDR3 comprising the amino acid sequence of SEQ ID NO:3, a heavy chain CDR1 comprising the amino acid sequence of SEQ ID NO:4, a heavy chain CDR2 comprising the amino acid sequence of SEQ ID NO:5, and a heavy chain CDR3 comprising the amino acid sequence of SEQ ID NO:6.

37. The use or composition of any one of claims 34-36, wherein the antibody or fragment thereof inhibits binding of IL-17A to said IL-17RA.

38. The use or composition of any one of claims 34-37, wherein the medicament or composition is for administration to a patient suffering from psoriasis.

39. The use or composition of any one of claims 34-38, wherein the medicament or composition is for administration to a patient suffering from plaque psoriasis, pustular psoriasis or psoriatic erythroderma.

40. The use or composition of claim 38 or 39, wherein the patient has a NAPSI score of at least 6 or a modified NAPSI score of at least 2 or 3 on one or more nails.

41. The use or composition of any one of claims 38-40, wherein the patient has a PSSI score of at least 15.

42. The use or composition of any one of claims 34-41, where the patient has a SSA score of at least 30%.

43. The use or composition of any one of claims 34-42, wherein the medicament or the composition comprises a dose of antibody or fragment thereof effective to reduce and maintain a NAPSI score of 6 or less or a mNAPSI score of 3 or less on an affected nail.

44. The use or composition of any one of claims 34-43, wherein the medicament or composition comprises a dose of antibody or fragment thereof effective to reduce and maintain a PSSI score of 15 or less.

45. The use or composition of any one of claims 34-44, wherein the medicament or composition comprises a dose of antibody that is 70 mg, 140mg, 210 mg or 280 mg.

46. The use or composition of any one of claims 34-45, wherein the medicament or composition is for administration to a patient having less than 10% body surface area affected by psoriasis and additionally having nail or scalp psoriasis.

47. The use or composition of any one of claims 34-46, wherein the medicament or composition is for administration to a patient having less than 10% body surface area affected by moderate to severe psoriasis, plaque psoriasis, pustular psoriasis or psoriatic erythroderma.

48. The use or composition of any one of claims 34-46, wherein the medicament or composition is administered with a second treatment.

49. The use or composition of claim 48, wherein said second treatment is administered prior to, concurrent with, or subsequent to administration of said medicament or composition comprising said antibody.

50. The use or composition of claim 48 or 49, wherein the second treatment is a topical treatment.

51. The use or composition of claim 50, where in the topical treatment is selected from the group consisting fluorouracil, dithranol, tazarotene, cyclosporine, calcineneurin inhibitors, triamcinolone, fluocinonide, topical steroids, vitamin D₃, vitamin D₃ analogs, betamethasone dipropionate, betamethasone valerate, calcipotriol, clobetasol, XAMIOL, DAIVOBET, coal tar, urea, corticosteroids, retinoids, anthralin, topical methatrexate, keratolytics, salicylic acid, tofacitinab, apremilast, topical JAK inhibitors or a combination thereof.

52. The use or composition of any one of claims 48-51, wherein the second treatment is selected from the group consisting of retinoids, acitretin cyclosporine, methotrexate, apremilast, tofacitinib, oral JAK inhibitors, oral PI3 kinase inhibitors, oral MAP kinase inhibitors, Fumaderm, fumarates, dimethyl fumarate, sulfasalazine, leflunomide, calcineurin inhibitors, azathioprine, thioguanine, hydroxyurea, hydroxychloroquine, sulfasalazine, antifungals or a combination thereof.

53. The use or composition of any one of claims 48-52, wherein the second treatment an antibody or chimeric protein specific for TNF, IL-17, IL-12/23 or IL-23.

54. The use or composition of claim 53, wherein the antibody or chimeric protein is infliximab, adalimumab, etanercept, alefacept, ustekinumab, secukinumab, ixekizumab, guselkumab, antifungals or a combination thereof.

55. The use or composition of any one of claims 48-54, wherein the second treatment is selected from the group consisting of is triamcinolone acetonide photochemotherapy, laser therapy, Excimer laser, oral/topical psorallen with UVA (PUVA), pulsed dye laser, radiation therapy, superficial radiotherapy, electron beam therapy, Grenz ray therapy, ermatome shaving, aloe vera extract, narrow band UV therapy UV therapy or a combination thereof.

56. The use or composition of any one of claims 34-55, wherein said antibody is selected from the group consisting of:
a. a humanized antibody;
b. a chimeric antibody;
c. a monoclonal antibody;
d. an antigen-binding antibody fragment;
e. a single chain antibody;
f. a diabody;
g. a triabody;
h. a tetrabody;
i. a Fab fragment;
j. a F(ab')2 fragment;
k. an IgD antibody;
l. an IgE antibody;
m. an IgM antibody;
n. an IgG1 antibody;
o. an IgG2 antibody;
p. an IgG3 antibody; and
q.an IgG4 antibody.

57. The use or composition of any one of claims 34-56, wherein said medicament or composition further comprises a pharmaceutically acceptable diluent.

58. The use or composition of any one of claims 34-57, wherein said medicament or composition comprises an aqueous solution of a glutamic acid buffer comprising a) said glutamic acid buffer comprises a glutamic acid concentration of 5-30 mM.+-.0.2 mM; b) said glutamic acid buffer comprises a pH of 4.5-5.2.+-.0.2; c) said formulation further comprises 2-4% proline (w/v) and 0.005-0.02% (w/v) polysorbate 20 and d) the antibody or fragment thereof at a concentration of 100 to 150 mg/ml.

59. The use or composition of claim 58, wherein the medicament or composition has an osmolarity of 275 to 325 osm.

60. The use or composition of claim 58 or 59, wherein the medicament or composition has a viscosity of 5 to 7 cP at 25° C.

61. The use or composition of any one of claims 34-60, wherein said antibody is a human IgG2 monoclonal antibody.
